(19) United States
(12) Patent Application Publication
(10) Pub. No.: US 2021/0187072 A1
(43) Pub. Date: Jun. 24, 2021

(54) METHODS AND COMPOSITIONS FOR REDUCING RISK OF RELAPSE AND PROLONGING SURVIVAL IN ACUTE MYELOID LEUKEMIA

(71) Applicants: Anna MARTNER, Gothenburg (SE); Fredrik Bergh THOREN, Gothenburg (SE); Kristoffer HELLSTRAND, Gothenburg (SE)

(72) Inventors: Anna MARTNER, Gothenburg (SE); Fredrik Bergh THOREN, Gothenburg (SE); Kristoffer HELLSTRAND, Gothenburg (SE)

(21) Appl. No.: 17/050,414

(22) PCT Filed: Jun. 20, 2019
(86) PCT No.: PCT/US2019/038172

§ 371 Date: Oct. 23, 2020

Related U.S. Application Data
(60) Provisional application No. 62/662,246, filed on Apr. 25, 2018.

(51) Int. Cl. A61K 38/20; A61K 31/417; A61K 45/06; A61P 35/02

(52) U.S. Cl. CPC A61K 38/2013; A61P 35/02; A61K 45/06; A61K 31/417

(57) ABSTRACT

Some embodiments provided herein relate to methods and compositions for reducing the risk of relapse of a hyperproliferative disorder, such as acute myeloid leukemia (AML), in a subject in which the hyperproliferative cells of the subject exhibit a normal karyotype and/or in a subject who has been administered no more than one induction therapeutic course. Some such embodiments include the administration of a NOX2 inhibitor, such as histamine dihydrochloride (HDC), in combination with a cytokine, such as interleukin-2 (L-2).

### CROSS-REFERENCE TO RELATED APPLICATIONS

This application claims the benefit of U.S. Prov. App. No. 62/662,246 entitled “NOX2 INHIBITORS AND LOW-DOSE INTERLEUKIN-2 FOR TREATING ACUTE MYELOID LEUKEMIA WITH NORMAL KARYOTYPE” filed Apr. 25, 2018 which is incorporated by reference in its entirety.

### FIELD OF THE DISCLOSURE

Some embodiments provided herein relate to methods and compositions for reducing the risk of relapse of a hyperproliferative disorder, such as acute myeloid leukemia (AML), in a subject in which the hyperproliferative cells of the subject exhibit a normal karyotype and/or in a subject who has been administered no more than one course of induction chemotherapy. Some such embodiments include the administration of a NOX2 inhibitor, such as histamine dihydrochloride (HDC), in combination with a cytokine, such as interleukin-2 (IL-2).

### BACKGROUND

Hyperproliferative disorders encompass a broad range of both non-malignant and malignant disorders in which cell growth is increased over normal levels. Acute myeloid leukemia (AML) is a genetically and morphologically heterogeneous leukemia in which myeloid cells expand in bone marrow and other organs. Most patients with AML achieve complete remission (CR) that can be identified as microscopic disappearance of leukemic cells, after initial induction rounds of chemotherapy, which are typically given immediately after diagnosis. The standard treatment in AML comprises additional consolidation rounds of chemotherapy aimed at eliminating residual leukemic cells. However, as many as 60% or more of adult patients will experience relapse of leukemia within 2-3 years, with poor prognosis for long-term survival. Relapse is a significant reason why the 5-year survival rate in adult AML remains in the range of 25-30% (Burnett et al., 2011 J Clin Oncol. 29:487-94). Accordingly, there remains a need for improved methods for treating, ameliorating and reducing the risk of relapse for hyperproliferative disorders, such as normal karyotype AML.

### SUMMARY OF CERTAIN INVENTIVE ASPECTS

Some embodiments include a method of reducing the risk of relapse for a hyperproliferative disorder in a subject, wherein a hyperproliferative cell of the subject comprises a normal karyotype, the method comprising: administering to the subject an effective amount of a nicotinamide adenine dinucleotide phosphate oxidase 2 (NOX2) inhibitor in combination with an effective amount of a cytokine, wherein the risk of relapse in the subject is reduced compared to an untreated subject not administered the NOX2 inhibitor in combination with the cytokine.

Some embodiments include a method of increasing a survival rate of a subject having a hyperproliferative disorder, wherein a hyperproliferative cell of the subject comprises a normal karyotype, the method comprising: administering to the subject an effective amount of a nicotinamide adenine dinucleotide phosphate oxidase 2 (NOX2) inhibitor in combination with an effective amount of a cytokine, wherein the survival rate of the subject is increased compared to an untreated subject not administered the NOX2 inhibitor in combination with the cytokine.

Some embodiments include a method of reducing the risk of relapse for a hyperproliferative disorder in a subject, wherein the subject has been administered no more than one induction chemotherapy course to treat the hyperproliferative disorder, the method comprising: administering to the subject an effective amount of a nicotinamide adenine dinucleotide phosphate oxidase 2 (NOX2) inhibitor in combination with an effective amount of a cytokine, wherein the risk of relapse in the subject is reduced compared to an untreated subject not administered the NOX2 inhibitor in combination with the cytokine.

Some embodiments include a method of increasing a survival rate of a subject having a hyperproliferative disorder, wherein the subject has been administered no more than one induction chemotherapy course to treat the hyperproliferative disorder, the method comprising: administering to the subject an effective amount of a nicotinamide adenine dinucleotide phosphate oxidase 2 (NOX2) inhibitor in combination with an effective amount of a cytokine, wherein the survival rate of the subject is increased compared to an untreated subject not administered the NOX2 inhibitor in combination with the cytokine.

In some embodiments, the subject is in complete remission (CR) from the hyperproliferative disorder. In some embodiments, the CR comprises less than 5% blast cells in normocellular bone marrow and an absence of extramedullary leukemia.

In some embodiments, the NOX2 inhibitor is selected from the group consisting of histamine, a histamine salt, histamine dihydrochloride (HDC), histamine diphosphate, a histamine structural analog having H2-receptor activities, an endogenous histamine releasing preparation, a non-histamine derivative H2-receptor agonist, GSK2795039, apocynin, diphenylene iodonium, GKT136901, GKT137831, ML171, VAS2870, VAS3947, celastrol, ebselen, perhexiline, grindelic acid, NOX2ds-tat, NOXA1ds, fulvene-5, ACD084, NSC23766, CAS 1177865-17-6, CAS 1090893-12-1, and shionogi. In some embodiments, the NOX2 comprises histamine dihydrochloride (HDC).

In some embodiments, the HDC is administered at a dosage of about 0.5 mg twice a day.

In some embodiments, the cytokine is selected from the group consisting of interleukin-2 (IL-2), modified variants of IL-2, interleukin-15 (IL-15), interleukin-18 (IL-18), interferon-alpha, interferon-beta, and interferon-gamma. In some embodiments, the cytokine is IL-2.

In some embodiments, the IL-2 is administered in an amount of about 5,000 U/kg/day to about 300,000 U/kg/day. In some embodiments, the IL-2 is administered at a dosage of 16,400 U/kg twice a day.

In some embodiments, the NOX2 inhibitor and the cytokine are administered sequentially. In some embodiments, the NOX2 inhibitor is administered prior to the cytokine. In some embodiments, the NOX2 inhibitor is administered after the cytokine.

In some embodiments, the NOX2 inhibitor and the cytokine are administered concurrently.

In some embodiments, the administration comprises a first cycle comprising daily administration of the NOX2 inhibitor in combination of the cytokine for 3 weeks, and no administration of the NOX2 inhibitor in combination of the cytokine for the following 3 weeks. In some embodiments, the first cycle is repeated for three cycles.

Some embodiments also include a second cycle comprising daily administration of the NOX2 inhibitor in combination of the cytokine for 3 weeks, and no administration of the NOX2 inhibitor in combination of the cytokine for the following 6 weeks. In some embodiments, the second cycle is repeated from six cycles.

In some embodiments, the hyperproliferative disorder is a leukemia. In some embodiments, the hyperproliferative disorder is selected from the group consisting of chronic myeloid leukemia, acute myeloid leukemia (AML), acute lymphocytic leukemia, T-cell acute lymphoblastic leukemia (T-ALL), and chronic lymphocytic leukemia. In some embodiments, the hyperproliferative disorder is acute myeloid leukemia (AML).

In some embodiments, a hyperproliferative cell of the subject comprises a normal karyotype. In some embodiments, the normal karyotype lacks at least a chromosomal duplication, a chromosomal deletion, a chromosomal insertion, a chromosomal inversion, or a chromosomal translocation. In some embodiments, the normal karyotype lacks at least one chromosomal aberration selected from a chromosome 5 deletion, a chromosome 7 deletion, a chromosome 8 duplication, a chromosome 21 duplication, a chromosome 22 duplication, a del(5q) deletion, a del(7q) deletion, a del(9q) deletion, a t(8;21) translocation, a t(9;11) translocation, a t(15;17) translocation, and an inv(16) inversion.

Some embodiments also include identifying a normal karyotype in the hyperproliferative cell of the subject. In some embodiments, the identification comprises obtaining a chromosomal spread of the hyperproliferative cell of the subject. In some embodiments, the identification comprises performing fluorescent in situ hybridization (FISH) on the hyperproliferative cell of the subject.

In some embodiments, administration of the NOX2 inhibitor in combination of the cytokine increases the survival rate of the subject by at least 10% compared to a survival rate for the untreated subject. In some embodiments, administration of the NOX2 inhibitor in combination of the cytokine increases the survival rate of the subject by at least 30% compared to a survival rate for the untreated subject. In some embodiments, administration of the NOX2 inhibitor in combination of the cytokine increases the survival rate of the subject by at least 50% compared to a survival rate for the untreated subject.

In some embodiments, administration of the NOX2 inhibitor in combination of the cytokine increases the survival rate of the subject compared to a survival rate for the untreated subject, wherein the survival rate is a leukemia-free survival rate.

In some embodiments, administration of the NOX2 inhibitor in combination of the cytokine increases the survival rate of the subject compared to a survival rate for the untreated subject, wherein the survival rate is an overall survival rate.

In some embodiments, administration of the NOX2 inhibitor in combination of the cytokine delays relapse of the hyperproliferative disorder in the subject by at least 3 months compared to a delay in relapse for the untreated subject. In some embodiments, administration of the NOX2 inhibitor in combination of the cytokine delays relapse of the hyperproliferative disorder in the subject by at least 6 months compared to a delay in relapse for the untreated subject. In some embodiments, administration of the NOX2 inhibitor in combination of the cytokine delays relapse of the hyperproliferative disorder in the subject by at least 12 months compared to a delay in relapse for the untreated subject.

In some embodiments, administration of the NOX2 inhibitor in combination of the cytokine reduces the risk of relapse of the hyperproliferative disorder for the subject by at least 10% compared to a risk of relapse for the untreated subject. In some embodiments, administration of the NOX2 inhibitor in combination of the cytokine reduces the risk of relapse of the hyperproliferative disorder for the subject by at least 30% compared to a risk of relapse for the untreated subject. In some embodiments, administration of the NOX2 inhibitor in combination of the cytokine reduces the risk of relapse of the hyperproliferative disorder for the subject by at least 50% compared to a risk of relapse for the untreated subject.

In some embodiments, a relapse of the hyperproliferative disorder comprises at least 5% blast cells in the bone marrow.

In some embodiments, a relapse of the hyperproliferative disorder comprises extramedullary leukemia.

In some embodiments, the subject has been administered no more than one induction chemotherapy course to treat the hyperproliferative disorder.

In some embodiments, the subject has not been administered consolidation therapy. In some embodiments, the consolidation therapy comprises immunotherapy, a graft, or chemotherapy.

In some embodiments, the subject is less than 60 years old.

In some embodiments, the subject is mammalian. In some embodiments, the subject is human.

Some embodiments include a kit for reducing the risk of relapse for a hyperproliferative disorder in a subject or for increasing a survival rate of the subject, comprising: a nicotinamide adenine dinucleotide phosphate oxidase 2 (NOX2) inhibitor; a cytokine; and a probe to identify a normal karyotype in a hyperproliferative cell.

In some embodiments, the NOX2 inhibitor is selected from the group consisting of histamine, a histamine salt, histamine dihydrochloride (HDC), histamine diphosphate, a histamine structural analog having H2-receptor activities, an endogenous histamine releasing preparation, a non-histamine derivative H2-receptor agonist, GSK2795039, apocynin, diphenylene iodonium, GKT136901, GKT137831, ML171, VAS2870, VAS3947, celastrol, ebselen, perhexiline, grindelic acid, NOX2ds-tat, NOXA1ds, fulvene-5, ACD084, NSC23766, CAS 1177865-17-6, CAS 1090893-12-1, and shionogi. In some embodiments, the NOX2 comprises histamine dihydrochloride (HDC).

In some embodiments, the cytokine is selected from the group consisting of interleukin-2 (IL-2), interleukin-15 (IL-15), interleukin-18 (IL-18), interferon-alpha, interferon-beta, and interferon-gamma. In some embodiments, the cytokine is IL-2.

In some embodiments, the probe to identify a normal karyotype in a hyperproliferative cell is selected from a chromosome-specific probe, or a chromosome stain selected from giemsa, mepacrine, DAPI and Hoescht 33258.

Some embodiments include use of a nicotinamide adenine dinucleotide phosphate oxidase 2 (NOX2) inhibitor in combination with a cytokine to reduce the risk of relapse for a hyperproliferative disorder in a subject or to increase a survival rate of the subject, wherein a hyperproliferative cell of the subject comprises a normal karyotype, wherein the risk of relapse in the subject is reduced compared to an untreated subject not administered the NOX2 inhibitor in combination with the cytokine.

Some embodiments include use of a nicotinamide adenine dinucleotide phosphate oxidase 2 (NOX2) inhibitor in combination with a cytokine in the preparation of a medicament to reduce the risk of relapse for a hyperproliferative disorder in a subject or to increase a survival rate of the subject, wherein a hyperproliferative cell of the subject comprises a normal karyotype, wherein the risk of relapse in the subject is reduced compared to an untreated subject not administered the NOX2 inhibitor in combination with the cytokine.

Some embodiments include use of a nicotinamide adenine dinucleotide phosphate oxidase 2 (NOX2) inhibitor in combination with a cytokine to reduce the risk of relapse for a hyperproliferative disorder in a subject or to increase a survival rate of the subject, wherein the subject has been administered no more than one induction chemotherapy course to treat the hyperproliferative disorder, wherein the risk of relapse in the subject is reduced compared to an untreated subject not administered the NOX2 inhibitor in combination with the cytokine.

Some embodiments include use of a nicotinamide adenine dinucleotide phosphate oxidase 2 (NOX2) inhibitor in combination with a cytokine in the preparation of a medicament to reduce the risk of relapse for a hyperproliferative disorder in a subject or to increase a survival rate of the subject, wherein the subject has been administered no more than one induction chemotherapy course to treat the hyperproliferative disorder, wherein the risk of relapse in the subject is reduced compared to an untreated subject not administered the NOX2 inhibitor in combination with the cytokine.

In some embodiments, the subject is in complete remission (CR) from the hyperproliferative disorder. In some embodiments, the CR comprises less than 5% blast cells in normocellular bone marrow and an absence of extramedullary leukemia.

In some embodiments, the NOX2 inhibitor is selected from the group consisting of histamine, a histamine salt, histamine dihydrochloride (HDC), histamine diphosphate, a histamine structural analog having H2-receptor activities, an endogenous histamine releasing preparation, a non-histamine derivative H2-receptor agonist, GSK2795039, apocynin, diphenylene iodonium, GKT136901, GKT137831, ML171, VAS2870, VAS3947, celastrol, ebselen, perhexiline, grindelic acid, NOX2ds-tat, NOXA1ds, fulvene-5, ACD084, NSC23766, CAS 1177865-17-6, CAS 1090893-12-1, and shionogi. In some embodiments, the NOX2 comprises histamine dihydrochloride (HDC).

In some embodiments, the HDC is administered at a dosage of about 0.5 mg twice a day.

In some embodiments, the cytokine is selected from the group consisting of interleukin-2 (IL-2), modified variants of IL-2, interleukin-15 (IL-15), interleukin-18 (IL-18), interferon-alpha, interferon-beta, and interferon-gamma. In some embodiments, the cytokine is IL-2.

In some embodiments, the IL-2 is administered in an amount of about 5,000 U/kg/day to about 300,000 U/kg/day. In some embodiments, the IL-2 is administered at a dosage of 16,400 U/kg twice a day.

In some embodiments, the NOX2 inhibitor and the cytokine are administered sequentially.

In some embodiments, the NOX2 inhibitor and the cytokine are administered concurrently.

In some embodiments, the administration comprises a first cycle comprising daily administration of the NOX2 inhibitor in combination of the cytokine for 3 weeks, and no administration of the NOX2 inhibitor in combination of the cytokine for the following 3 weeks. In some embodiments, the first cycle is repeated for three cycles.

Some embodiments also include a second cycle comprising daily administration of the NOX2 inhibitor in combination of the cytokine for 3 weeks, and no administration of the NOX2 inhibitor in combination of the cytokine for the following 6 weeks In some embodiments, the second cycle is repeated from six cycles.

In some embodiments, the hyperproliferative disorder is a leukemia. In some embodiments, the hyperproliferative disorder is selected from the group consisting of chronic myeloid leukemia, acute myeloid leukemia (AML), acute lymphocytic leukemia, T-cell acute lymphoblastic leukemia (T-ALL), and chronic lymphocytic leukemia. In some embodiments, the hyperproliferative disorder is acute myeloid leukemia (AML).

In some embodiments, a hyperproliferative cell of the subject comprises a normal karyotype. In some embodiments, the normal karyotype lacks at least a chromosomal duplication, a chromosomal deletion, a chromosomal insertion, a chromosomal inversion, or a chromosomal translocation. In some embodiments, the normal karyotype lacks at least one chromosomal aberration selected from a chromosome 5 deletion, a chromosome 7 deletion, a chromosome 8 duplication, a chromosome 21 duplication, a chromosome 22 duplication, a del(5q) deletion, a del(7q) deletion, a del(9q) deletion, a t(8;21) translocation, a t(9;11) translocation, a t(15;17) translocation, and an inv(16) inversion.

Some embodiments also include identifying a normal karyotype in the hyperproliferative cell of the subject. In some embodiments, the identification comprises obtaining a chromosomal spread of the hyperproliferative cell of the subject. In some embodiments, the identification comprises performing fluorescent in situ hybridization (FISH) on the hyperproliferative cell of the subject.

In some embodiments, administration of the NOX2 inhibitor in combination of the cytokine increases the survival rate of the subject by at least 10% compared to a survival rate for the untreated subject. In some embodiments, administration of the NOX2 inhibitor in combination of the cytokine increases the survival rate of the subject by at least 30% compared to a survival rate for the untreated subject. In some embodiments, administration of the NOX2 inhibitor in combination of the cytokine increases the survival rate of the subject by at least 50% compared to a survival rate for the untreated subject.

In some embodiments, administration of the NOX2 inhibitor in combination of the cytokine increases the survival rate of the subject compared to a survival rate for the untreated subject, wherein the survival rate is a leukemia-free survival rate.

In some embodiments, administration of the NOX2 inhibitor in combination of the cytokine increases the survival rate of the subject compared to a survival rate for the untreated subject, wherein the survival rate is an overall survival rate.

In some embodiments, administration of the NOX2 inhibitor in combination of the cytokine delays relapse of the hyperproliferative disorder in the subject by at least 3 months compared to a delay in relapse for the untreated subject. In some embodiments, administration of the NOX2 inhibitor in combination of the cytokine delays relapse of the hyperproliferative disorder in the subject by at least 6 months compared to a delay in relapse for the untreated subject. In some embodiments, administration of the NOX2 inhibitor in combination of the cytokine delays relapse of the hyperproliferative disorder in the subject by at least 12 months compared to a delay in relapse for the untreated subject.

In some embodiments, administration of the NOX2 inhibitor in combination of the cytokine reduces the risk of relapse of the hyperproliferative disorder for the subject by at least 10% compared to a risk of relapse for the untreated subject. In some embodiments, administration of the NOX2 inhibitor in combination of the cytokine reduces the risk of relapse of the hyperproliferative disorder for the subject by at least 30% compared to a risk of relapse for the untreated subject. In some embodiments, administration of the NOX2 inhibitor in combination of the cytokine reduces the risk of relapse of the hyperproliferative disorder for the subject by at least 50% compared to a risk of relapse for the untreated subject.

In some embodiments, a relapse of the hyperproliferative disorder comprises at least 5% blast cells in the bone marrow.

In some embodiments, a relapse of the hyperproliferative disorder comprises extramedullary leukemia.

In some embodiments, the subject has been administered no more than one induction chemotherapy course to treat the hyperproliferative disorder.

In some embodiments, the subject has not been administered consolidation chemotherapy. In some embodiments, the consolidation therapy comprises immunotherapy, a graft, or chemotherapy.

In some embodiments, the subject is less than 60 years old.

In some embodiments, the subject is mammalian. In some embodiments, the subject is human.

Some embodiments include a method of treating a hyperproliferative disorder in a subject in which the hyperproliferative cells of said subject exhibit a normal karyotype comprising the steps of administering a first composition comprising an agent selected from the group consisting of histamine, a histamine structural analog having H2 receptor activities, an endogenous histamine releasing preparation and a non-histamine derivative H2 receptor agonist to said subject; and administering a second composition comprising a cytokine to said subject.

Some embodiments include a method of reducing the tumor burden in a subject with primary or metastatic cancer in which the tumor cells of said subject exhibit a normal karyotype comprising the steps of administering a first composition comprising an agent selected from the group consisting of histamine, a histamine structural analog having H2 receptor activities, an endogenous histamine releasing preparation and a non-histamine derivative H2 receptor agonist to said subject; and administering a second composition comprising a cytokine to said subject.

Some embodiments include a method of reducing the risk of metastatic tumor spread in a subject with active cancer in which the tumor cells of said subject exhibit a normal karyotype comprising the steps of administering a first composition comprising an agent selected from the group consisting of histamine, a histamine structural analog having H2 receptor activities, an endogenous histamine releasing preparation and a non-histamine derivative H2 receptor agonist to said subject; and administering a second composition comprising a cytokine to said subject.

Some embodiments include a method of preventing or delaying the reappearance, recurrence or metastatic spread of cancer in a subject in which the cancer cells of said subject exhibit a normal karyotype comprising the steps of administering a first composition comprising an agent selected from the group consisting of histamine, a histamine structural analog having H2 receptor activities, an endogenous histamine releasing preparation and a non-histamine derivative H2 receptor agonist to said subject; and administering a second composition comprising a cytokine to said subject.

Some embodiments include a method of preventing relapse to a cancer in a subject in which the cancer cells of said subject exhibit a normal karyotype comprising the steps of administering a first composition comprising an agent selected from the group consisting of histamine, a histamine structural analog having H2 receptor activities, an endogenous histamine releasing preparation and a non-histamine derivative H2 receptor agonist to said subject; and administering a second composition comprising a cytokine to said subject.

Some embodiments include a method of delaying the relapse to a cancer in a subject in remission from said cancer in which the cancer cells of said subject exhibit a normal karyotype comprising the steps of administering a first composition comprising an agent selected from the group consisting of histamine, a histamine structural analog having H2 receptor activities, an endogenous histamine releasing preparation and a non-histamine derivative H2 receptor agonist to said subject; and administering a second composition comprising a cytokine to said subject.

Some embodiments include a method of prolonging the remission from a cancer in a subject in which the cancer cells of said subject exhibit a normal karyotype comprising the steps of administering a first composition comprising an agent selected from the group consisting of histamine, a histamine structural analog having H2 receptor activities, an endogenous histamine releasing preparation and a non-histamine derivative H2 receptor agonist to said subject; and administering a second composition comprising a cytokine to said subject.

Some embodiments include a method of increasing the survival of a subject in remission from a cancer in which the cancer cells of said subject exhibit a normal karyotype comprising the steps of administering a first composition comprising an agent selected from the group consisting of histamine, a histamine structural analog having H2 receptor activities, an endogenous histamine releasing preparation and a non-histamine derivative H2 receptor agonist to said subject; and administering a second composition comprising a cytokine to said subject.

Some embodiments include a method of prolonging the survival time of a subject in remission from a cancer in which the cancer cells of said subject exhibit a normal karyotype comprising the steps of administering a first composition comprising an agent selected from the group consisting of histamine, a histamine structural analog having H2 receptor activities, an endogenous histamine releasing preparation and a non-histamine derivative H2 receptor agonist to said subject; and administering a second composition comprising a cytokine to said subject.

Some embodiments include a method of reducing malignant tumor growth in a subject in which the tumor cells of said subject exhibit a normal karyotype comprising the steps of administering a first composition comprising an agent selected from the group consisting of histamine, a histamine structural analog having H2 receptor activities, an endogenous histamine releasing preparation and a non-histamine derivative H2 receptor agonist to said subject; and administering a second composition comprising a cytokine to said subject.

Some embodiments include a method of decreasing or alleviating cancer symptoms in a subject in remission from a cancer in which the cancer cells of said subject exhibit a normal karyotype comprising the steps of administering a first composition comprising an agent selected from the group consisting of histamine, a histamine structural analog having H2 receptor activities, an endogenous histamine releasing preparation and a non-histamine derivative H2 receptor agonist to said subject; and administering a second composition comprising a cytokine to said subject.

In some embodiments, said hyperproliferative disorder comprises cancer. In some embodiments, said cancer comprises leukemia. In some embodiments, said leukemia comprises acute myeloid leukemia (AML). In some embodiments, said leukemia comprises T-cell acute lymphoblastic leukemia (T-ALL). In some embodiments, said cancer comprises a solid tumor. In some embodiments, said cancer comprises a B-cell lymphoma or a thymoma. In some embodiments, said hyperproliferative disorder comprises a pre-cancerous condition or a benign proliferative disorder.

In some embodiments, said histamine receptor agonist comprises histamine dihydrochloride.

In some embodiments, said histamine dihydrochloride is administered at a dose of 0.5 mg.

In some embodiments, said histamine receptor agonist comprises N-methyl-histamine or 4-methyl-histamine.

In some embodiments, said cytokine comprises interleukin-2 (IL-2). In some embodiments, said IL-2 is administered at a dosage of 16,400 U/kg twice a day.

In some embodiments, said first composition, said second composition, or both of said compositions are intravenously administered to said subject.

In some embodiments, said first composition, said second composition, or both of said compositions are orally administered to said subject.

In some embodiments, said first composition, said second composition, or both of said compositions are subcutaneously administered to said subject.

In some embodiments, said first composition and said second composition are administered together in a single composition.

In some embodiments, said first composition and said second composition are administered at separate sites.

In some embodiments, said first composition and said second composition are administered at separate times.

In some embodiments, said first composition, said second composition, or both of said compositions are administered once a day.

In some embodiments, said first composition, said second composition, or both of said compositions are administered twice a day.

In some embodiments, said subject is in complete remission (CR) from said hyperproliferative disorder.

Some embodiments include a kit for treating or ameliorating a hyperproliferative disorder in a subject in which the hyperproliferative cells of said subject exhibit a normal karyotype comprising a first composition comprising an agent selected from the group consisting of histamine, a histamine structural analog having H2 receptor activities, an endogenous histamine releasing preparation and a non-histamine derivative H2 receptor agonist; and a second composition comprising a cytokine.

### BRIEF DESCRIPTION OF THE DRAWINGS

FIG. 1A depicts a Kaplan-Meier analysis for leukemia-free survival (LFS) in the first complete remission (CR1) for AML subjects (n=97) identified to have leukemic cells of non-normal karyotypes, and treated with histamine dihydrochloride (HDC) in combination with IL-2, or control (no treatment) after the completion of consolidation chemotherapy. Subjects were identified to have structural chromosomal changes in leukemic cells. The clinical outcome was compared using the log rank test.

FIG. 1B depicts a Kaplan-Meier analysis for overall survival (OS) in the first complete remission (CR1) for AML subjects (n=97) identified to have leukemic cells of non-normal karyotypes, and treated with HDC in combination with IL-2, or control (no treatment) after the completion of consolidation chemotherapy. Subjects were identified to have structural chromosomal changes in leukemic cells. The clinical outcome was compared using the log rank test.

FIG. 2A depicts a Kaplan-Meier analysis for leukemia-free survival (LFS) in the first complete remission (CR1) for AML subjects (n=128) identified to have leukemic cells of normal karyotype, and treated with HDC in combination with IL-2 or control (no treatment) after the completion of consolidation chemotherapy. Subjects were identified to have no structural chromosomal changes in leukemic cells. The clinical outcome was compared using the log rank test.

FIG. 2B depicts a Kaplan-Meier analysis for overall survival (OS) in the first complete remission (CR1) for AML subjects (n=128) identified to have leukemic cells of normal karyotype, and treated with HDC in combination with IL-2, or control (no treatment) after the completion of consolidation chemotherapy. Subjects were identified to have no structural chromosomal changes in leukemic cells. The clinical outcome was compared using the log rank test.

FIG. 3A depicts a Kaplan-Meier analysis for leukemia-free survival (LFS) in the first complete remission (CR1) for AML subjects (n=72) less than 60 years old, identified to have leukemic cells of normal karyotype, and treated with HDC in combination with IL-2 or control (no treatment) after the completion of consolidation chemotherapy. Subjects were identified to have no structural chromosomal changes in leukemic cells. The clinical outcome was compared using the log rank test.

FIG. 3B depicts a Kaplan-Meier analysis for overall survival (OS) in the first complete remission (CR1) for AML subjects (n=72) less than 60 years old, identified to have leukemic cells of normal karyotype, and treated with HDC in combination with IL-2 or control (no treatment) after the completion of consolidation chemotherapy. Subjects were identified to have no structural chromosomal changes in leukemic cells. The clinical outcome was compared using the log rank test.

FIG. 4A depicts a Kaplan-Meier analysis for leukemia-free survival (LFS) in the first complete remission (CR1) for AML subjects (n=203) regardless of age, having attained complete remission after a single induction therapy, and treated with HDC in combination with IL-2, or control (no treatment) after the completion of consolidation chemotherapy.

FIG. 4B depicts a Kaplan-Meier analysis for overall survival (OS) in the first complete remission (CR1) for AML subjects (n=203) regardless of age, having attained complete remission after a single induction therapy, and treated with HDC in combination with IL-2 or control (no treatment) after the completion of consolidation chemotherapy.

FIG. 4C depicts a Kaplan-Meier analysis for leukemia-free survival (LFS) in the first complete remission (CR1) for AML subjects (n=130) less than 60 years old, having attained complete remission after a single induction therapy, and treated with HDC in combination with IL-2 or control (no treatment) after the completion of consolidation chemotherapy.

FIG. 4D depicts a Kaplan-Meier analysis for overall survival (OS) in the first complete remission (CR1) for AML subjects (n=130) less than 60 years old, having attained complete remission after a single induction therapy, and treated with HDC in combination with IL-2, or control (no treatment) after the completion of consolidation chemotherapy,

FIG. 4E depicts a Kaplan-Meier analysis for leukemia-free survival (LFS) in the first complete remission (CR1) for AML subjects (n=48) less than 60 years old diagnosed with AML of the M4 or M5 FAB classification, having attained complete remission after a single induction therapy, and treated with HDC in combination with IL-2, or control (no treatment) after the completion of consolidation chemotherapy.

FIG. 4F depicts a Kaplan-Meier analysis for overall survival (OS) in the first complete remission (CR1) for AML subjects (n=48) less than 60 years old diagnosed with AML of the M4 or M5 FAB classification, having attained complete remission after a single induction therapy, and treated with HDC in combination with IL-2 or control (no treatment) after the completion of consolidation chemotherapy.

FIG. 4G depicts a Kaplan-Meier analysis for leukemia-free survival (LFS) in the first complete remission (CR1) for AML subjects (n=96) less than 60 years old diagnosed with AML of all FAB classes except M2 (“non-M2”) having attained complete remission after a single induction therapy, and treated with HDC in combination with IL-2 or control (no treatment) after the completion of consolidation chemotherapy.

FIG. 4H depicts a Kaplan-Meier analysis for overall survival (OS) in the first complete remission (CR1) for AML subjects (n=96) less than 60 years old diagnosed with AML of all FAB classes except M2 (“non-M2”) having attained complete remission after a single induction therapy, and treated with HDC in combination with IL-2 or control (no treatment) after the completion of consolidation chemotherapy.

FIG. 4I depicts a Kaplan-Meier analysis for leukemia-free survival (LFS) in the first complete remission (CR1) for AML subjects (n=57) regardless of age having attained complete remission after two or more courses of induction therapy, and treated with HDC in combination with IL-2 or control (no treatment) after the completion of consolidation chemotherapy.

FIG. 4J depicts a Kaplan-Meier analysis for overall survival (OS) in the first complete remission (CR1) for AML subjects (n=57) regardless of age having attained complete remission after a single induction therapy, and treated with HDC in combination with IL-2 or control (no treatment) after the completion of consolidation chemotherapy.

### DETAILED DESCRIPTION

Some embodiments provided herein relate to methods and compositions for reducing the risk of relapse of a hyperproliferative disorder, such as acute myeloid leukemia (AML), in a subject in which the hyperproliferative cells of the subject exhibit a normal karyotype and/or in a subject who has been administered no more than one induction therapeutic course. Some such embodiments include the administration of a NOX2 inhibitor, such as histamine dihydrochloride (HDC), in combination with a cytokine, such as interleukin-2 (IL-2).

Hyperproliferative disorders such as AML can be characterized by rapid clonal expansion of immature myeloid cells in bone marrow. After initial rounds of induction chemotherapy, a majority of patients can attain complete remission, such as leukemic cells constitute less than 5% of nucleated cells in the bone marrow along with the return of non-malignant hematopoiesis. In younger patients, such as those under 60, but also in older patients who are deemed fit to tolerate intensive chemotherapy, the induction chemotherapy typically includes treatment with cytarabine (ara-C), and an anthracycline such as daunorubicin (daunomycin) or idarubicin. The most common form of induction chemotherapy is sometimes called the 7+3 regimen, because it includes the administration of cytarabine continuously for 7 days along with an anthracycline on each of the first 3 days. Other chemotherapeutics, alone or in combination, may however be administered to AML patients aiming to achieve complete remission. Approximately 60-70% of AML patients who receive induction chemotherapy will achieve complete remission. In remaining patients, one or several additional courses of induction chemotherapy are required.

After achieving complete remission, patients may receive additional courses of consolidation therapy aiming at eliminating residual leukemia. However, approximately 60-70% of adult patients in first complete remission (CR1) experience relapse of AML, mostly within 2-3 years, with dismal prognosis for long-term survival. The absence of efficient means to prevent relapse in the post-chemotherapy phase in non-transplanted patients is a significant reason why the rate of 5-year survival in adult AML is in the range of 25-30%.

AML cells can include somatic genetic aberrations that may be grouped by chromosomal morphology into AML of aberrant or normal karyotype. In approximately 55% of human adult AML, the leukemic cells thus carry structurally or numerically aberrant chromosomes, including deletion or multiplication of chromosomes or chromosome sections, such as −5, −7, +8, +21, +22, del(5q), del(7q), del(9q), or inverted/translocated chromosome arms or segments, such as t(8;21), t(9;11), t(15;17), inv(16). Normal karyotype AML, where the chromosomes of leukemic cells are morphologically and numerically intact, can include mutations in certain genes, such as NPM1, FLT3-ITD, CEBPA, DNMT3A, IDH1 and IDH2 and constitute approximately 45% of all adult AML.

Aspects of lymphocyte function and phenotype have been reported to impact on the risk of relapse in the post-consolidation phase of AML, and several immunotherapies have been developed aiming at reducing the relapse risk. These include administration of antibodies against leukemia-associated antigens, treatment with immunostimulatory cytokines, adoptive transfer of anti-leukemic lymphocytes and vaccine strategies using dendritic cells carrying leukemic antigens or fused with autologous leukemic cells. Of these, immunotherapy with HDC and low-dose IL-2 has proven efficacious in terms of relapse prevention in a randomized phase III setting.

Treatment with HDC/IL-2 can include targeting the formation of immunosuppressive reactive oxygen species produced by the NOX2 enzyme of myeloid cells (HDC component), while concomitantly activating and expanding populations of natural killer (NK) cells and T cells (IL-2 component). These components act in synergy to promote NK and T cell function and viability.

Embodiments of the methods and compositions provided herein relate to the unexpected observation that the structure of chromosomes in cancer cells from patients with AML can determine the clinical efficacy of immunotherapy with HDC and low-dose IL-2. Specifically, as disclosed herein the leukemia-free survival and overall survival of adult AML patients was favorably impacted by treatment with HDC/IL-2 in patients in whom cancer cells were devoid of structural changes in chromosomes (‘normal karyotype AML’) but absent in cases of AML where any chromosomal alteration was observed (aberrant karyotype).

### Certain Methods and Compositions

Some embodiments of the methods and compositions provided herein include treating a hyperproliferative disorder in a subject, in which the hyperproliferative cells of said subject exhibit a normal karyotype, comprising the steps of administering a first composition comprising an agent selected from the group consisting of histamine, a histamine structural analog having agonistic H2 receptor activities, an endogenous histamine releasing preparation and a non-histamine derivative H2 receptor agonist to said subject; and administering a second composition comprising a cytokine to said subject.

Some embodiments of the methods and compositions provided herein include reducing the tumor burden in a subject with primary or metastatic cancer in which the tumor cells of said subject exhibit a normal karyotype comprising the steps of administering a first composition comprising an agent selected from the group consisting of histamine, a histamine structural analog having agonistic H2 receptor activities, an endogenous histamine releasing preparation and a non-histamine derivative H2 receptor agonist to said subject; and administering a second composition comprising a cytokine to said subject.

Some embodiments of the methods and compositions provided herein include reducing the risk of metastatic tumor spread in a subject with active cancer in which the tumor cells of said subject exhibit a normal karyotype comprising the steps of administering a first composition comprising an agent selected from the group consisting of histamine, a histamine structural analog having agonistic H2 receptor activities, an endogenous histamine releasing preparation and a non-histamine derivative H2 receptor agonist to said subject; and administering a second composition comprising a cytokine to said subject.

Some embodiments of the methods and compositions provided herein include preventing or delaying the reappearance, recurrence or metastatic spread of cancer in a subject in which the cancer cells of said subject exhibit a normal karyotype comprising the steps of administering a first composition comprising an agent selected from the group consisting of histamine, a histamine structural analog having agonistic H2 receptor activities, an endogenous histamine releasing preparation and a non-histamine derivative H2 receptor agonist to said subject; and administering a second composition comprising a cytokine to said subject.

Some embodiments of the methods and compositions provided herein include preventing relapse of a cancer in a subject in which the cancer cells of said subject exhibit a normal karyotype comprising the steps of administering a first composition comprising an agent selected from the group consisting of histamine, a histamine structural analog having agonistic H2 receptor activities, an endogenous histamine releasing preparation and a non-histamine derivative H2 receptor agonist to said subject; and administering a second composition comprising a cytokine to said subject.

Some embodiments of the methods and compositions provided herein include delaying the relapse to a cancer in a subject in remission from said cancer in which the cancer cells of said subject exhibit a normal karyotype comprising the steps of administering a first composition comprising an agent selected from the group consisting of histamine, a histamine structural analog having agonistic H2 receptor activities, an endogenous histamine releasing preparation and a non-histamine derivative H2 receptor agonist to said subject; and administering a second composition comprising a cytokine to said subject.

Some embodiments of the methods and compositions provided herein include prolonging the remission from a cancer in a subject in which the cancer cells of said subject exhibit a normal karyotype comprising the steps of administering a first composition comprising an agent selected from the group consisting of histamine, a histamine structural analog having agonistic H2 receptor activities, an endogenous histamine releasing preparation and a non-histamine derivative H2 receptor agonist to said subject; and administering a second composition comprising a cytokine to said subject.

Some embodiments of the methods and compositions provided herein include increasing the survival of a subject in remission from a cancer in which the cancer cells of said subject exhibit a normal karyotype comprising the steps of administering a first composition comprising an agent selected from the group consisting of histamine, a histamine structural analog having agonistic H2 receptor activities, an endogenous histamine releasing preparation and a non-histamine derivative H2 receptor agonist to said subject; and administering a second composition comprising a cytokine to said subject.

Some embodiments of the methods and compositions provided herein include prolonging the survival time of a subject in remission from a cancer in which the cancer cells of said subject exhibit a normal karyotype comprising the steps of administering a first composition comprising an agent selected from the group consisting of histamine, a histamine structural analog having agonistic H2 receptor activities, an endogenous histamine releasing preparation and a non-histamine derivative H2 receptor agonist to said subject; and administering a second composition comprising a cytokine to said subject.

Some embodiments of the methods and compositions provided herein include reducing malignant tumor growth in a subject in which the tumor cells of said subject exhibit a normal karyotype comprising the steps of administering a first composition comprising an agent selected from the group consisting of histamine, a histamine structural analog having agonistic H2 receptor activities, an endogenous histamine releasing preparation and a non-histamine derivative H2 receptor agonist to said subject; and administering a second composition comprising a cytokine to said subject.

Some embodiments of the methods provided herein include a method of decreasing or alleviating cancer symptoms in a subject in remission from a cancer in which the cancer cells of said subject exhibit a normal karyotype comprising the steps of administering a first composition comprising an agent selected from the group consisting of histamine, a histamine structural analog having agonistic H2 receptor activities, an endogenous histamine releasing preparation and a non-histamine derivative H2 receptor agonist to said subject; and administering a second composition comprising a cytokine to said subject.

### Histamine

In some embodiments, a hyperproliferative disorder treatment comprises administration of histamine in combination with one or more therapeutic agents as described herein. In some embodiments, a histamine structural analog comprises H2 receptor agonists. In some embodiments, an endogenous histamine releasing preparation is used in the methods and compositions provided herein. In some embodiments, a non-histamine derivative H2 receptor agonist is used in the methods and compositions provided herein. In some embodiments, the histamine receptor agonist comprises histamine dihydrochloride. In some embodiments, the histamine dihydrochloride is administered at a dose of 0.5 mg. In some embodiments, the histamine receptor agonist comprises N-methyl-histamine. In some embodiments, the histamine receptor agonist comprises Nu-methyl-histamine dihydrochloride (NMH). In some embodiments, the histamine receptor agonist comprises 4-methyl-histamine. In some embodiments, the histamine comprises other histamine H2-receptor agonists. HDC is commercially available and methods of making HDC as well as other forms of histamine are known in the art. See e.g. U.S. Pat. No. 6,528,654, which is incorporated herein by reference.

### Cytokines

In some embodiments, a cytokine is used in the methods and compositions provided herein. In some embodiments, the cytokine is an interleukin. In some embodiments, the interleukin comprises IL-2, interleukin-15 (IL-15) or interleukin-18 (IL-18). In some embodiments, the IL-2 is an IL-2 variant having increased potency and/or binding to an IL-2 receptor, compared to a wild-type IL-2 (See. e.g, Klein C., et al., (2013) Blood 122:2278; Levin A M. et al., (2012) Cancer Discovery 2:388; and Klein C., et al (2017) Oncoimmunology. 6:e1277306). In some embodiments, the interleukin is administered at a low dose. In some embodiments, IL-2, IL-15 or IL-18 are administered at a dosage of 16,400 U/kg two times per day or at higher doses.

In some embodiments, the cytokine is an interferon. In some embodiments, the interferon is interferon-alpha. In some embodiments, the interferon is interferon-beta. In some embodiments, the interferon is interferon-gamma.

In some embodiments, the cytokine is a hematopoietic growth factor. In some embodiments, the hematopoietic growth factor is selected from the group consisting of: Erythropoietin, IL-11, Granulocyte-macrophage colony-stimulating factor (GM-CSF), and Granulocyte colony-stimulating factor (G-CSF), or a combination thereof.

In some embodiments, the cancer treatment comprises administering HDC and IL-2. In some embodiments, HDC administered at 0.5 mg two times per day and IL-2 administered at 16,400 U/kg two times per day, as described in herein, was highly and unexpectedly effective in achieving overall survival (OS) and leukemia free survival (LFS) in patients of all ages having normal karyotype AML.

In some embodiments, each treatment cycle lasts 3 weeks. In some embodiments, each treatment cycle lasts 1-5 weeks. In some embodiments, each treatment cycle lasts 1 week. In some embodiments, each treatment cycle lasts 2 weeks. In some embodiments, each treatment cycle lasts 4 weeks.

In some embodiments, the post-consolidation therapy comprises 10 cycles. In some embodiments, the consolidation therapy comprises 1-5 cycles. In some embodiments, the consolidation therapy comprises 5-10 cycles. In some embodiments, the consolidation therapy comprises 10-15 cycles. In some embodiments, the consolidation therapy comprises 5 cycles. In some embodiments, the consolidation therapy comprises 15 cycles. In some embodiments, the consolidation therapy comprises 7 cycles. In some embodiments, the consolidation therapy comprises 12 cycles. In some embodiments, the consolidation therapy comprises 3 cycles.

In some embodiments, the samples from the subject are tissue samples. In some embodiments, the samples from the subject are blood samples. In some embodiments, the blood sample is a peripheral blood sample. In some embodiments, the samples from the subject are cerebrospinal fluid (CSF) samples. In some embodiments, the samples from the subject are urine samples. In some embodiments, the samples from the subject are fecal samples.

### Certain Modes of Administration

In some embodiments, the administration of the composition comprising histamine, a histamine structural analog having agonistic H2 receptor activity, an endogenous histamine releasing preparation, or a non-histamine derivative H2 receptor agonist and the composition comprising a cytokine may occur either simultaneously or time-staggered, either at the same site of administration or at different sites of administration. In some embodiments, the first composition and the second composition are administered together in a single composition. In some embodiments, the first composition and the second composition are administered at separate sites.

In some embodiments, the first composition and the second composition are administered at separate times. In some embodiments, the first composition, the second composition, or both the first and second composition are administered once a day.

In some embodiments, the first composition, the second composition, or both the first and second compositions are administered twice a day.

In some embodiments, any of the therapeutic or prophylactic drugs or compounds described herein may be administered simultaneously. In some embodiments, they may be administered at different time than one another. In some embodiments, they may be administered within a few minutes of one another. In some embodiments, they may be administered within a few hours of one another. In some embodiments, they may be administered within 1 hour of one another. In some embodiments, they may be administered within 2 hours of one another. In some embodiments, they may be administered within 5 hours of one another. In some embodiments, they may be administered within 12 of one another. In some embodiments, they may be administered within 24 hours of one another.

In some embodiments, any of the therapeutic or prophylactic drugs or compounds described herein may be administered at the same site of administration. In some embodiments, they may be administered at different sites of administration.

In some embodiments, the first composition is intravenously administered to the subject. In some embodiments, the second composition is intravenously administered to the subject. In some embodiments, both the first and the second compositions are intravenously administered to the subject.

In some embodiments, the first composition is orally administered to the subject. In some embodiments, the second composition is orally administered to the subject. In some embodiments, both the first and the second compositions are orally administered to the subject.

In some embodiments, the first composition is subcutaneously administered to the subject. In some embodiments, the second composition is subcutaneously administered to the subject. In some embodiments, both the first and the second compositions are subcutaneously administered to the subject.

### Hyperproliferative Disorders

Some embodiments provided herein include methods of treating a hyperproliferative disorder in a subject in which the hyperproliferative cells of the subject exhibit a normal karyotype. In some embodiments, the hyperproliferative disorder comprises a pre-cancerous condition. In some embodiments, the hyperproliferative disorder comprises a benign proliferative disorder. In some embodiments, the subject is in complete remission (CR) from the hyperproliferative disorder.

In some embodiments, the hyperproliferative disorder comprises a cancer. In some embodiments, the cancer comprises leukemia. In some embodiments, the leukemia comprises AML. In some embodiments, the leukemia comprises T-cell acute lymphoblastic leukemia (T-ALL). In some embodiments, the cancer comprises a solid tumor. In some embodiments, the cancer comprises a T-cell lymphoma, thymoma, a B-cell leukemia or a B-cell lymphoma.

In some embodiments, methods of treating cancer as described herein comprise methods of preventing or delaying relapse to a cancer in a subject in remission, prolonging remission from cancer, increasing survival, decreasing or alleviating cancer symptoms, or a combination thereof. In some embodiments, methods of treating cancer as described herein comprise methods of reducing the tumor burden in a subject with primary or metastatic cancer in a subject, reducing the risk of metastatic tumor spread in a subject with active cancer, preventing or delaying the reappearance, recurrence or metastatic spread of cancer in a subject, preventing relapse to a cancer in a subject, delaying the relapse to a cancer in a subject in remission from said cancer, prolonging the remission from a cancer in a subject, increasing the survival of a subject in remission from a cancer, prolonging the survival time of a subject in remission from a cancer, reducing malignant tumor growth in a subject, decreasing or alleviating cancer symptoms in a subject in remission from a cancer, or a combination thereof. In some embodiments, the cancer cells of the subject exhibit a normal karyotype.

In some embodiments, a subject treated by the methods of the present disclosure has or is suffering from a cancer or tumor. In some embodiments, a subject treated by the methods provided herein is in remission from a cancer or tumor. In some embodiments, the subject is in complete remission (CR). In some embodiments, the CR is from leukemia. Methods of evaluating complete remission are well known in the art.

In some embodiments, a subject treated by the methods provided herein has a solid tumor or lymphoma and showed a “response” to earlier cancer treatment. In some embodiments, a subject treated by the methods provided herein has a solid tumor or lymphoma and showed a “complete response” to earlier cancer treatment.

In some embodiments, the subject having cancer or a tumor has been treated with surgery, chemotherapy, radiation therapy, a targeted therapy, including immunotherapies that are intended to boost immune system responses against cancer, or a combination thereof.

In some embodiments, the subject in the methods provided herein has or had cancer. In some embodiments, the subject has or had a tumor. In some embodiments, the subject has a neoplastic disease. In some embodiments, the subject has a malignancy. In some embodiments, the subject has or had a pre-cancerous condition or a pre-malignant condition. In some embodiments, the subject has or had another hyperproliferative disorder.

In some embodiments, the cancer is a leukemia. In some embodiments, the leukemia is AML. In some embodiments, the leukemia is chronic myeloid leukemia (CML). In some embodiments, the leukemia is chronic myelomonocytic leukemia (CMML). In some embodiments, the leukemia is acute lymphocytic leukemia (ALL). In some embodiments, the leukemia is chronic lymphocytic leukemia (CLL). In some embodiments, the leukemia is hairy cell leukemia.

In some embodiments, the tumor is a solid tumor. In some embodiments, the solid tumor is a colon carcinoma, prostate cancer, breast cancer, lung cancer, skin cancer, liver cancer, bone cancer, ovary cancer, pancreas cancer, brain cancer, head and neck cancer or other solid tumor.

In some embodiments, the cancer or tumor is in the breast, prostate, lung, colon, stomach, pancreas, ovary, or brain. In some embodiments, the cancer is a hematopoietic cancer, a neuroblastoma, or a malignant glioma.

In some embodiments, the cancer is selected from one or more of the following: Adrenocortical Carcinoma, AIDS-Related Cancers, Kaposi Sarcoma, AIDS-Related Lymphoma, Primary CNS Lymphoma, Anal Cancer, Appendix Cancer, Astrocytomas, Atypical Teratoid/Rhabdoid Tumor, Central Nervous System Tumors, Basal Cell Carcinoma and other forms of Skin Cancer (Nonmelanoma), Bile Duct Cancer, Bladder Cancer, Bone Cancer, Ewing Sarcoma Family of Tumors, Osteosarcoma and Malignant Fibrous Histiocytoma, Brain Stem Glioma, Brain Tumor, Astrocytomas, Brain and Spinal Cord Tumors Treatment Overview, Brain Stem Glioma, Central Nervous System Atypical Teratoid/Rhabdoid Tumor, Central Nervous System Embryonal Tumors, Central Nervous System Germ Cell Tumors, Craniopharyngioma, Ependymoma, Breast Cancer, Bronchial Tumors, Burkitt Lymphoma and other forms of Non-Hodgkin Lymphoma, Carcinoid Tumor, Gastrointestinal Carcinoma of Unknown Primary Origin, Cardiac (Heart) Tumors, Central Nervous System, Atypical Teratoid/Rhabdoid Tumor, Embryonal Tumors, Germ Cell Tumor, Lymphoma, Primary, Cervical Cancer, Cholangiocarcinoma, Chordoma, Chronic Myeloproliferative Neoplasms, Colon Cancer, Colorectal Cancer, Craniopharyngioma, Cutaneous T-Cell Lymphoma including Mycosis Fungoides and Sezary Syndrome, Ductal Carcinoma In Situ (DCIS), Embryonal Tumors, Central Nervous System, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Germ Cell Tumor, Central Nervous System, Extracranial, Extragonadal, Ovarian, Testicular, Gestational Trophoblastic Disease, Glioma—see Brain Tumor Brain Stem, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumors, Pancreatic Neuroendocrine Tumors, Kaposi Sarcoma, Kidney, Renal Cell, Wilms Tumor and Other Childhood Kidney Tumors, Langerhans Cell Histiocytosis, Laryngeal Cancer, Acute Lymphoblastic (ALL), Acute Myeloid (AML), Chronic Lymphocytic (CLL), Chronic Myelogenous (CML), Hairy Cell, Lip and Oral Cavity Cancer, Liver Cancer (Primary), Lung Cancer, Non-Small Cell, Small Cell, Lymphoma, AIDS-Related Burkitt and other forms of Non-Hodgkin Lymphoma, Cutaneous T-Cell Cancer, Mycosis Fungoides and Sezary Syndrome, Hodgkin, Non-Hodgkin, Primary Central Nervous System (CNS), Macroglobulinemia Waldenström, Male Breast Cancer, Malignant Fibrous Histiocytoma of Bone and Osteosarcoma, Melanoma, Intraocular (Eye), Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myeloma, Multiple, Myeloproliferative Neoplasms, Chronic, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and, Oropharyngeal Cancer, Osteosarcoma and Malignant Fibrous Histiocytoma of Bone, Ovarian Cancer, Epithelial, Germ Cell Tumor, Low Malignant Potential Tumor, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma, Ewing, Kaposi, Osteosarcoma (Bone Cancer), Rhabdomyosarcoma, Soft Tissue, Uterine, Vascular Tumors, Sezary Syndrome, Skin Cancer, Melanoma, Merkel Cell Carcinoma, Nonmelanoma, Small Cell Lung Cancer, Small Intestine Cancer, Soft Tissue Sarcoma, Squamous Cell Carcinoma—see Skin Cancer (Nonmelanoma), Squamous Neck Cancer with Occult Primary, Metastatic, Stomach (Gastric) Cancer, T-Cell Lymphoma, Cutaneous—see Mycosis Fungoides and Sezary Syndrome, Testicular Cancer, Throat Cancer, Thymoma and Thymic Carcinoma, Thyroid Cancer, Transitional Cell Cancer of the Renal Pelvis and Ureter, Primary Carcinoma, Ureter and Renal Pelvis, Transitional Cell Cancer, Urethral Cancer, Uterine Cancer, Endometrial, Uterine Sarcoma, Vaginal Cancer, Vascular Tumors, Vulvar Cancer, Waldenström Macroglobulinemia, and Wilms Tumor.

In some embodiments, the pre-cancerous condition is actinic keratosis, Barrett's esophagus, atrophic gastritis, ductal carcinoma in situ, dyskeratosis congenita, sideropenic dysphagia, lichen planus, oral submucous fibrosis, solar elastosis, cervical dysplasia, leukoplakia, erythroplakia, or a combination thereof. In some embodiments, the pre-cancerous condition is a dysplasia or a benign neoplasia. In some embodiments, the pre-cancerous condition is prostatic intraepithelial neoplasia (PIN), proliferative inflammatory atrophy (PIA), atypical small acinar proliferation (ASAP), squamous intraepithelial lesion (SIL), Atypical endometrial hyperplasia, ovarian epithelial dysplasia, Breast calcifications, MGUS (monogammopathy of unknown significance), Vulval intra-epithelial neoplasia (VIN), Lobular carcinoma in situ (LCIS) Vaginal intra-epithelial neoplasia, or VAIN Vulval lichen sclerosus and lichen planus, Cervical intra-epithelial neoplasia (CIN), Barrett's oesophagus, or a combination thereof.

In some embodiments, the cancer treatment described in the methods provided herein is given to a subject in remission. In some embodiments, the subject is in remission from a hematopoietic cancer. In some embodiments, it is given to a subject who has undergone induction therapy. In some embodiments, induction chemotherapy comprises a combination of cytarabine and daunorubicin. In some embodiments, it is given to a subject who has completed consolidation therapy. In some embodiments, consolidation chemotherapy comprises a combination of cytarabine and daunorubicin. In some embodiments, the consolidation phase comprises 2-4 courses of high-dose cytarabine, sometimes with the addition of an anthracyline (in some embodiments, daunorubicin or, in some embodiments, idarubicin). In some embodiments, the consolidation phase comprises high-dose cytarabine without the addition of anthracyclines. In some embodiments, the consolidation phase comprises an allogeneic or autologous transplant. In some embodiments, the subject receives an allogeneic or autologous transplant after the completion of the consolidation phase.

In some embodiments, the cancer treatment is an immunological treatment or an immunotherapy. In some embodiments, the treatment strengthens the immune response in the subject to the cancer. In some embodiments, the treatment induces an immune response in the subject to the cancer. In some embodiments, the treatment comprises administration of a cytokine to the subject. In some embodiments, the cytokine is an immunostimulant.

As used herein, “hyperproliferative disorder” can include a disorder that results from a hyperproliferation of cells. Example hyperproliferative disorders include to cancer or autoimmune diseases. Other hyperproliferative disorders may include vascular occlusion, restenosis, atherosclerosis, or inflammatory bowel disease.

As used herein, “hyperproliferative disease” refers to conditions wherein cell growth is increased over normal levels. For example, hyperproliferative diseases or disorders include malignant diseases (e.g., esophageal cancer, colon cancer, biliary cancer) and non-malignant diseases (e.g., atherosclerosis, benign hyperplasia, and benign prostatic hypertrophy). Further examples of a hyperproliferative disorder and/or hyperproliferative tissue/cell types include melanoma, lymphoma (e.g., Hodgkin lymphoma, non-Hodgkin lymphoma, a B-cell neoplasm, a T-cell neoplasm, an NK cell neoplasm), leukemia, reticuloendothelial hyperplasia (e.g., reticuloendothelial neoplasia), lymphatic neoplasia, hematopoietic neoplasia, myeloma, multiple myeloma, an immunodeficiency-associated lymphoproliferative disorder, adenoma, adenocarcinoma, sarcoma (non-limiting examples of which include a lymphosarcoma, liposarcoma, osteosarcoma, chondrosarcoma, leiomyosarcoma, rhabdomyosarcoma, fibrosarcoma, the like or combinations thereof), carcinoma, breast cancer, colorectal cancer, gastrointestinal cancer, hepatocellular cancer, lung cancer, bone cancer, renal cancer, bladder cancer, hepatoma, neuroblastoma, retinoblastoma, astrocytoma, glioma, glioblastoma, medulloblastoma, meningioma, oligodendrocytoma, cervical cancer, testicular cancer, ovarian cancer, mesothelioma, esophageal cancer, pancreatic cancer, prostate cancer, the like or combinations thereof. A hyperproliferative disorder may be benign, malignant, metastatic, non-metastatic or undetermined.

As used herein, the terms “cytogenetically normal acute myeloid leukemia (CN-AML)” and “acute myelogenous leukemia with normal karyotype” are used interchangeably to refer to normal karyotype acute myeloid leukemia (NK-AML).

### Systems and Kits

Some embodiments of the methods and compositions provided herein include kits and systems. Some embodiments include a nicotinamide adenine dinucleotide phosphate oxidase 2 (NOX2) inhibitor; a cytokine; and a probe to identify a normal karyotype in a hyperproliferative cell. In some embodiments, the NOX2 inhibitor is selected from the group consisting of histamine, a histamine salt, HDC, histamine diphosphate, a histamine structural analog having H2-receptor activities, an endogenous histamine releasing preparation, a non-histamine derivative H2-receptor agonist, GSK2795039, apocynin, diphenylene iodonium, GKT136901, GKT137831, ML171, VAS2870, VAS3947, celastrol, ebselen, perhexiline, grindelic acid, NOX2ds-tat, NOXA1ds, fulvene-5, ACD084, NSC23766, CAS 1177865-17-6, CAS 1090893-12-1, and shionogi. In some embodiments, the NOX2 comprises HDC. In some embodiments, the cytokine is selected from the group consisting of IL-2, IL-15, IL-18, interferon-alpha, interferon-beta, and interferon-gamma. In some embodiments, the cytokine is IL-2. In some embodiments, the probe to identify a normal karyotype in a hyperproliferative cell is selected from a chromosome-specific probe, or a chromosome stain selected from giemsa, mepacrine, DAPI and Hoescht 33258.

Some such kits and systems can include a kit for treating a hyperproliferative disorder in a subject in which the hyperproliferative cells of said subject exhibit a normal karyotype comprising a first composition comprising an agent selected from the group consisting of histamine, a histamine structural analog having H2 receptor activities, an endogenous histamine releasing preparation and a non-histamine derivative H2 receptor agonist; and a second composition comprising a cytokine, and instructions for the use of said kit.

Some embodiments include a kit for reducing the tumor burden in a subject with primary or metastatic cancer in which the tumor cells of said subject exhibit a normal karyotype comprising a first composition comprising an agent selected from the group consisting of histamine, a histamine structural analog having agonistic H2 receptor activities, an endogenous histamine releasing preparation and a non-histamine derivative H2 receptor agonist; and a second composition comprising a cytokine, and instructions for the use of said kit.

Some embodiments include a kit for reducing the risk of metastatic tumor spread in a subject with active cancer in which the tumor cells of said subject exhibit a normal karyotype comprising a first composition comprising an agent selected from the group consisting of histamine, a histamine structural analog having agonistic H2 receptor activities, an endogenous histamine releasing preparation and a non-histamine derivative H2 receptor agonist; and a second composition comprising a cytokine, and instructions for the use of said kit.

Some embodiments include a kit for preventing or delaying the reappearance, recurrence or metastatic spread of cancer in a subject in which the cancer cells of said subject exhibit a normal karyotype comprising a first composition comprising an agent selected from the group consisting of histamine, a histamine structural analog having agonistic H2 receptor activities, an endogenous histamine releasing preparation and a non-histamine derivative H2 receptor agonist; and a second composition comprising a cytokine, and instructions for the use of said kit.

Some embodiments include a kit for preventing relapse to a cancer in a subject in which the cancer cells of said subject exhibit a normal karyotype comprising a first composition comprising an agent selected from the group consisting of histamine, a histamine structural analog having agonistic H2 receptor activities, an endogenous histamine releasing preparation and a non-histamine derivative H2 receptor agonist; and a second composition comprising a cytokine, and instructions for the use of said kit.

Some embodiments include a kit for delaying the relapse to a cancer in a subject in remission from said cancer in which the cancer cells of said subject exhibit a normal karyotype comprising a first composition comprising an agent selected from the group consisting of histamine, a histamine structural analog having agonistic H2 receptor activities, an endogenous histamine releasing preparation and a non-histamine derivative H2 receptor agonist; and a second composition comprising a cytokine, and instructions for the use of said kit.

Some embodiments include a kit for prolonging the remission from a cancer in a subject in which the cancer cells of said subject exhibit a normal karyotype comprising a first composition comprising an agent selected from the group consisting of histamine, a histamine structural analog having agonistic H2 receptor activities, an endogenous histamine releasing preparation and a non-histamine derivative H2 receptor agonist; and a second composition comprising a cytokine, and instructions for the use of said kit.

Some embodiments include a kit for increasing the survival of a subject in remission from a cancer in which the cancer cells of said subject exhibit a normal karyotype comprising a first composition comprising an agent selected from the group consisting of histamine, a histamine structural analog having H2 agonistic receptor activities, an endogenous histamine releasing preparation and a non-histamine derivative H2 receptor agonist; and a second composition comprising a cytokine, and instructions for the use of the kit.

Some embodiments include a kit for prolonging the survival time of a subject in remission from a cancer in which the cancer cells of said subject exhibit a normal karyotype comprising a first composition comprising an agent selected from the group consisting of histamine, a histamine structural analog having H2 agonistic receptor activities, an endogenous histamine releasing preparation and a non-histamine derivative H2 receptor agonist; and a second composition comprising a cytokine, and instructions for the use of said kit.

Some embodiments include a kit for reducing malignant tumor growth in a subject in which the tumor cells of said subject exhibit a normal karyotype comprising a first composition comprising an agent selected from the group consisting of histamine, a histamine structural analog having agonistic H2 receptor activities, an endogenous histamine releasing preparation and a non-histamine derivative H2 receptor agonist; and a second composition comprising a cytokine, and instructions for the use of said kit.

Some embodiments include a kit for decreasing or alleviating cancer symptoms in a subject in remission from a cancer in which the cancer cells of said subject exhibit a normal karyotype comprising a first composition comprising an agent selected from the group consisting of histamine, a histamine structural analog having agonistic H2 receptor activities, an endogenous histamine releasing preparation and a non-histamine derivative H2 receptor agonist; and a second composition comprising a cytokine, and instructions for the use of said kit.

### Methods of Treatment

In some embodiments, a cancer treatment of the present disclosure prevents relapse to a cancer. In some embodiments, a cancer treatment provided herein prevents recurrence of a cancer. In some embodiments, a cancer treatment provided herein delays the relapse of a cancer. In some embodiments, a cancer treatment provided herein delays the recurrence of a cancer. In some embodiments, a cancer treatment provided herein prolongs remission from a cancer. In some embodiments, a cancer treatment provided herein increases the survival of a subject with cancer. In some embodiments, a cancer treatment provided herein prolongs the survival time of a subject with cancer. In some embodiments, a cancer treatment provided herein treats cancer in a subject. In some embodiments, a cancer treatment provided herein decreases cancer symptoms in a subject. In some embodiments, a cancer treatment provided herein alleviates cancer symptoms in a subject. In some embodiments, a cancer treatment provided herein prolongs cancer-free remission. In some embodiments, a cancer treatment provided herein reduces the size of malignant tumors in a subject. In some embodiments, a cancer treatment provided herein reduces the tumor burden in patients with primary or metastatic cancer. In some embodiments, a cancer treatment provided herein reduces the risk of metastatic tumor spread in patients with active cancer. In some embodiments, a cancer treatment provided herein prevents or delays the reappearance, recurrence or metastatic spread of cancer in patients who have undergone surgery, chemotherapy or any other treatment to reduce the cancer burden.

In some embodiments, remission comprises remission from hematopoietic cancer. In some embodiments, remission from hematopoietic cancer is when subjects are microscopically free of cancer cells (in bone marrow, blood or other organs) along with the re-appearance of normal hematopoiesis, In some embodiments, remission has been achieved after induction chemotherapy.

In some embodiments, cancer remission is a decrease in or disappearance of signs and symptoms of cancer. In some embodiments, the remission is a partial remission, which in some embodiments, is when some, but not all, signs and symptoms of cancer have disappeared. In some embodiments, the remission is a complete remission, which in some embodiments is when all signs and symptoms of cancer have disappeared, although cancer still may be in the body.

In some embodiments, a subject goes into (achieves) remission after induction therapy, which in some embodiments comprises surgery, chemotherapy or other means of reducing a tumor burden.

In some embodiments, “treating” refers to both therapeutic treatment and prophylactic or preventative measures, wherein the object is to prevent or lessen the targeted pathologic condition or disorder as described herein. Therefore, some embodiments comprise administering to a subject while the subject is in remission from cancer after chemotherapy or surgery or other treatments. Some embodiments comprise administering to a subject who has relapsed to cancer. Some comprise administering to a subject who has active cancer.

In some embodiments, treating may include directly affecting or curing, suppressing, inhibiting, preventing, reducing an incidence, reducing the severity of, delaying the onset of, reducing symptoms associated with the disease, disorder or condition, or a combination thereof. Thus, in some embodiments, “treating” refers inter alia to delaying progression, expediting remission, inducing remission, augmenting remission, speeding recovery, increasing efficacy of or decreasing resistance to alternative therapeutics, or a combination thereof. In some embodiments, treating refers to reducing the pathogenesis of, ameliorating the symptoms of, ameliorating the secondary symptoms of, or prolonging the latency to a relapse of a cancer in a subject. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

Some embodiments include alleviating symptoms in a subject. In some embodiments, symptoms are primary, while in some embodiments, symptoms are secondary. In some embodiments, “primary” refers to a symptom that is a direct result of the cancer, while in some embodiments, “secondary” refers to a symptom that is derived from or consequent to a primary cause. In some embodiments, the methods of the present disclosure treat primary or secondary symptoms or secondary complications related to cancer.

In some embodiments, “symptoms” may be any manifestation of a cancer, including twitching, cramping, stiffness of muscles; muscle weakness affecting an arm or a leg; slurred or nasal speech; difficulty chewing or swallowing; general weakness, atrophy, or a combination thereof.

In some embodiments, the subject is human. In some embodiments, the subject is a non-human primate. In some embodiments, the subject is murine, which In some embodiments is a mouse, and, In some embodiments is a rat. In some embodiments, the subject is canine, feline, bovine, equine, laprine or porcine. In some embodiments, the subject is mammalian. In some embodiments, the subject is any organism susceptible to cancer or tumors.

### Certain Therapeutic Compositions and Methods

Some embodiments of the methods and compositions provided herein n comprise administering a pharmaceutical composition comprising a therapeutic agent as described herein, including histamine, a histamine structural analog having agonistic H2 receptor activities, an endogenous histamine releasing preparation, a non-histamine derivative H2 receptor agonist, or a cytokine, and a pharmaceutically acceptable carrier.

As used herein, “pharmaceutical composition” can include a therapeutically effective amount of the active ingredient, i.e. histamine, a histamine structural analog having agonistic H2 receptor activities, an endogenous histamine releasing preparation, a non-histamine derivative H2 receptor agonist or a cytokine, together with a pharmaceutically acceptable carrier or diluent. As used herein, “therapeutically effective amount” or “pharmaceutically effective amount” can include an amount of therapeutic agent, which has a therapeutic effect. The dosages of a pharmaceutically active ingredient which are useful in treatment are therapeutically effective amounts. Thus, as used herein, a therapeutically effective amount means those amounts of therapeutic agent which produce the desired therapeutic effect as judged by clinical trial results and/or model animal studies.

In some embodiments, the pharmaceutical compositions containing the therapeutic agent can be administered to a subject by any method known to a person skilled in the art, such as parenterally, transmucosally, transdermally, intramuscularly, intravenously, intra-dermally, intra-peritonealy, intra-ventricularly, intra-cranially, intra-vaginally or intra-tumorally. In some embodiments, the therapeutic agent is administered subcutaneously.

In some embodiments as described in the methods and compositions provided herein, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In some embodiments provided herein, the active ingredient is formulated in a capsule. In accordance with this embodiment, the compositions provided herein comprise, in addition to the active compound and the inert carrier or diluent, a hard gelating capsule.

In some embodiments, the pharmaceutical compositions are administered by intravenous, intra-arterial, or intra-muscular injection of a liquid preparation. Suitable liquid formulations include solutions, suspensions, dispersions, emulsions, oils and the like. In some embodiments, the pharmaceutical compositions are administered intravenously and are thus formulated in a form suitable for intravenous administration. In some embodiments, the pharmaceutical compositions are administered intra-arterially and are thus formulated in a form suitable for intra-arterial administration. In some embodiments, the pharmaceutical compositions are administered intra-muscularly and are thus formulated in a form suitable for intra-muscular administration.

In some embodiments, the pharmaceutical compositions are administered topically to body surfaces and are thus formulated in a form suitable for topical administration. Suitable topical formulations include gels, ointments, creams, lotions, drops and the like. For topical administration, the therapeutic agent is prepared and applied as a solution, suspension, or emulsion in a physiologically acceptable diluent with or without a pharmaceutical carrier.

In some embodiments, the pharmaceutical compositions provided herein are controlled-release compositions, i.e. compositions in which the therapeutic agent is released over a period of time after administration. Controlled- or sustained-release compositions include formulation in lipophilic depots (e.g. fatty acids, waxes, oils). In some embodiments, the composition is an immediate-release composition, i.e. a composition in which all of the therapeutic agent is released immediately after administration.

### Certain Therapeutic Embodiments

Some embodiments of the methods and compositions provided herein include reducing the risk of relapse for a hyperproliferative disorder in a subject, increasing a survival rate of a subject having a hyperproliferative disorder, and/or increasing a period of remission for a subject having a hyperproliferative disorder. In some embodiments the reduction or the increase in an attribute, such as risk, survival and/or remission, is with respect to an untreated subject. In some embodiments, the subject is in complete remission from the hyperproliferative disorder. In some embodiments, the complete remission comprises less than 5% blast cells in normocellular bone marrow and an absence of extramedullary leukemia.

In some such embodiments, the subject having the hyperproliferative disorder is a subject in which a karyotype of a hyperproliferative cell of the subject can comprise a normal karyotype. For example, the karyotype of a hyperproliferative cell can include a normal complement of chromosomes with no structural changes, and lacking any chromosomal duplication, a chromosomal deletion, a chromosomal insertion, a chromosomal inversion, and chromosomal translocation. In some embodiments, the normal karyotype lacks at least one chromosomal aberration selected from a chromosome 5 deletion, a chromosome 7 deletion, a chromosome 8 duplication, a chromosome 21 duplication, a chromosome 22 duplication, a del(5q) deletion, a del(7q) deletion, a del(9q) deletion, a t(8;21) translocation, a t(9;11) translocation, a t(15;17) translocation, and an inv(16) inversion. In some embodiments, the hyperproliferative cell of the subject can comprise a normal karyotype, yet can include other mutations, such as one or more mutations which may contribute to the cause of the hyperproliferative disorder. Some embodiments also include identifying a normal karyotype in the hyperproliferative cell of the subject. In some embodiments, the identification comprises obtaining a chromosomal spread of the hyperproliferative cell of the subject. In some embodiments, the identification comprises performing fluorescent in situ hybridization (FISH) on the hyperproliferative cell of the subject.

In some embodiments, the subject has been administered no more than a single course of induction therapy to treat the hyperproliferative disorder. In some such embodiments, the subject has not been administered a consolidation therapy to treat the hyperproliferative disorder.

Some embodiments of the methods and compositions provided herein for reducing the risk of relapse for a hyperproliferative disorder in a subject, in which a hyperproliferative cell of the subject comprises a normal karyotype, include administering to the subject an effective amount of a nicotinamide adenine dinucleotide phosphate oxidase 2 (NOX2) inhibitor in combination with an effective amount of a cytokine, wherein the risk of relapse in the subject is reduced compared to an untreated subject not administered the NOX2 inhibitor in combination with the cytokine. As used herein administration of a NOX2 inhibitor in combination with a cytokine can include administration of the NOX2 inhibitor and the cytokine such that both compounds are present in a subject at the same time. Thus, the NOX2 inhibitor and the cytokine can be administered concurrently or sequentially, at the same site in the subject, or different sites in the subject.

Some embodiments of the methods and compositions provided herein for increasing a survival rate of a subject having a hyperproliferative disorder, in which a hyperproliferative cell of the subject comprises a normal karyotype, include administering to the subject an effective amount of a NOX2 inhibitor in combination with an effective amount of a cytokine, wherein the survival rate of the subject is increased compared to an untreated subject not administered the NOX2 inhibitor in combination with the cytokine.

Some embodiments of the methods and compositions provided herein for reducing the risk of relapse for a hyperproliferative disorder in a subject, in which the subject has been administered no more than one induction chemotherapy course to treat the hyperproliferative disorder, include administering to the subject an effective amount of a NOX2 inhibitor in combination with an effective amount of a cytokine, wherein the risk of relapse in the subject is reduced compared to an untreated subject not administered the NOX2 inhibitor in combination with the cytokine.

Some embodiments of the methods and compositions provided herein for increasing a survival rate of a subject having a hyperproliferative disorder, in which the subject has been administered no more than one induction chemotherapy course to treat the hyperproliferative disorder, include administering to the subject an effective amount of a NOX2 inhibitor in combination with an effective amount of a cytokine, wherein the survival rate of the subject is increased compared to an untreated subject not administered the NOX2 inhibitor in combination with the cytokine.

In some embodiments, the NOX2 inhibitor is selected from the group consisting of histamine, a histamine salt, HDC, histamine diphosphate, a histamine structural analog having agonistic H2-receptor activities, an endogenous histamine releasing preparation, a non-histamine derivative H2-receptor agonist, GSK2795039, apocynin, diphenylene iodonium, GKT136901, GKT137831, ML171, VAS2870, VAS3947, celastrol, ebselen, perhexiline, grindelic acid, NOX2ds-tat, NOXA1ds, fulvene-5, ACD084, NSC23766, CAS 1177865-17-6, CAS 1090893-12-1, and shionogi. In some embodiments, the NOX2 comprises HDC.

In some embodiments, the NOX2 inhibitor is administered to the subject several times a day, twice a day, daily, several times a week, weekly, several times a month, or monthly. In some embodiments, the NOX2 inhibitor is administered to the subject twice daily. In some embodiments, the NOX2 inhibitor is administered at a dosage predicted to saturate about 70% to about 80% of phagocyte cell histamine H2 receptors in a subject. In some embodiments, the dosage of a NOX2 inhibitor, such as HDC is 0.001 mg, 0.01 mg, 0.05 mg, 0.1 mg, 0.2 mg, 0.3 mg, 0.4 mg, 0.5 mg, 0.6 mg, 0.7 mg, 0.8 mg, 0.9 mg, 1.0 mg, 2.0 mg, 3.0 mg, 4.0 mg, 5.0 mg, 6.0 mg, 7.0 mg, 8.0 mg, 9.0 mg, 10 mg, 20 mg, 50 mg, 100 mg, 200 mg, 500 mg, or a dosage between any two of the foregoing amounts. In some embodiments, the dosage of a NOX2 inhibitor, such as HDC is about 0.5 mg. In some embodiments, the dosage of a NOX2 inhibitor, such as HDC is 0.5 mg.

In some embodiments, the cytokine is selected from the group consisting of IL-2, IL-15, IL-18, interferon-alpha, interferon-beta, and interferon-gamma. In some embodiments, the cytokine is IL-2.

In some embodiments, the cytokine is administered to the subject several times a day, twice a day, daily, several times a week, weekly, several times a month, or monthly. In some embodiments, the cytokine is administered to the subject twice daily. In some embodiments, the cytokine, such as IL-2, is administered in an amount of about 5,000 U/kg/day to about 300,000 U/kg/day. In some embodiments, the IL-2 is administered at a dosage of 16,400 U/kg twice a day.

In some embodiments, the NOX2 inhibitor and the cytokine are administered sequentially. In some embodiments, the NOX2 inhibitor is administered prior to the cytokine. In some embodiments, the NOX2 inhibitor is administered after the cytokine. In some embodiments, the NOX2 inhibitor and the cytokine are administered concurrently. In some embodiments, the administration comprises a first cycle comprising daily administration of the NOX2 inhibitor in combination of the cytokine for 3 weeks, and no administration of the NOX2 inhibitor in combination of the cytokine for the following 3 weeks. In some embodiments, the first cycle is repeated for three cycles. Some embodiments also include a second cycle comprising daily administration of the NOX2 inhibitor in combination of the cytokine for 3 weeks, and no administration of the NOX2 inhibitor in combination of the cytokine for the following 6 weeks. In some embodiments, the second cycle is repeated from six cycles.

In some embodiments, the hyperproliferative disorder is a leukemia. In some embodiments, the hyperproliferative disorder is selected from the group consisting of chronic myeloid leukemia, AML, acute lymphocytic leukemia, T-cell acute lymphoblastic leukemia (T-ALL), and chronic lymphocytic leukemia. In some embodiments, the hyperproliferative disorder is AML.

In some embodiments, administration of the NOX2 inhibitor in combination of the cytokine increases the survival rate of the subject compared to a survival rate for the untreated subject by at least 10%, 20%, 30%, 40%, 50%, 60%, 70%, 80%, 90%, 100%, or any percentage between any two of the foregoing percentages.

In some embodiments, the survival rate is a leukemia-free survival rate. In some embodiments, the survival rate is an overall survival rate.

In some embodiments, administration of the NOX2 inhibitor in combination of the cytokine delays relapse of the hyperproliferative disorder in the subject compared to a delay in relapse for the untreated subject by at least 1 month, 2 months, 3 months, 6 months, 7 months, 8 months, 9 months, 10 months, 11 months, 12 months, or a period of time between any two of the foregoing periods.

In some embodiments, administration of the NOX2 inhibitor in combination of the cytokine reduces the risk of relapse of the hyperproliferative disorder for the subject compared to a risk of relapse for the untreated subject by at least 10%, 20%, 30%, 40%, 50%, 60%, 70%, 80%, 90%, 100%, or any percentage between any two of the foregoing percentages.

In some embodiments, a relapse of the hyperproliferative disorder comprises at least 5% blast cells in the bone marrow. In some embodiments, a relapse of the hyperproliferative disorder comprises extramedullary leukemia.

In some embodiments, the subject has been administered no more than one induction chemotherapy courses to treat the hyperproliferative disorder. In some embodiments, the subject has not been administered consolidation therapy. In some embodiments, the consolidation therapy comprises immunotherapy, a graft, or chemotherapy.

In some embodiments, the subject is less than 60 years old. In some embodiments, the subject is mammalian. In some embodiments, the subject is human.

### EXAMPLES

### Example 1—Phase III Clinical Trial with HDC/IL-2

### Methods

Three hundred twenty patients with AML (median age, 57 years; range, 18-84 years) were randomly assigned to treatment with HDC/IL-2 or no treatment (control) in a phase III trial. Patients were also classified as having AML using the French-American-British (FAB) classification (MO-M7). FAB classification included the following classes: MO: undifferentiated acute myeloblastic leukemia; M: acute myeloblastic leukemia with minimal maturation; M2: acute myeloblastic leukemia with maturation; M3: acute promyelocytic leukemia (APL); M4: acute myelomonocytic leukemia; M4 eos: acute myelomonocytic leukemia with eosinophilia; M5: acute monocytic leukemia; M6: acute erythroid leukemia; and M7: acute megakaryoblastic leukemia

The treatment was initiated after the completion of chemotherapy (induction and consolidation) and aimed to prevent of relapse of AML in the post-consolidation phase, which occurs in approximately 70% of adult AML. The treatment comprised ten 21-day cycles with IL-2 (16 400 U/kg) plus HDC (Ceplene) (0.5 mg). Study arms were balanced for age, sex, previous treatment, leukemic karyotypes, time from complete remission (CR) to inclusion and frequency of secondary leukemia. Details of treatment, stratification, toxicity and outcome are accounted for elsewhere (Brune et al., 2006 Blood 108(1):88-96, which is incorporated herein by reference in its entirety).

Briefly, patients in the treatment arm received 10 consecutive 3-week cycles of HDC/IL-2, whereas patients in the control arm received no treatment. The treatment continued for a total of 18 months or until the patients relapsed, died, discontinued therapy because of adverse events, withdrew consent, or became lost to follow-up. Cycles 1 to 3 comprised 3 weeks of treatment and 3 weeks off treatment, and in cycles 4 to 10 the off-treatment periods were extended to 6 weeks. In each cycle, patients in the treatment arm received HDC (Maxim Pharmaceuticals, San Diego, Calif.) at 0.5 mg subcutaneous twice a day and human recombinant IL-2 (aldesleukin; 16 400 U/kg subcutaneous twice a day; Chiron Corporation, Emeryville, Calif.). After 18 months of treatment (HDC/IL-2 arm) or observation (control arm), all patients were followed for at least 18 additional months until the study closure date. To avoid acute toxicity, HDC was administered at a rate not exceeding 0.1 mg per minute. In the event of HDC-related side effects, the injection time was prolonged to 7 to 10 minutes; if toxicity persisted, the dose was reduced by 20%. Reduction of the IL-2 dose was prescribed in case of side effects or inconveniences related to this treatment. The dose of HDC was predicted to saturate 70% to 80% of phagocyte cell histamine H2 receptors, and the dose of IL-2 had been shown previously to cause significant expansion of cytotoxic lymphocytes with documented antileukemic activity (See. e.g., Lanas A I, et al., 1994. Scand J Gastroenterol. 29: 678-683; and Meropol N J, et al. 1996 Clin Cancer Res. 2:669-677). The doses and schedules were considered suitable for long-term treatment on the basis of the results obtained in a pilot study.

Complete remission (CR) was defined as less than 5% blast cells in normocellular bone marrow, without evidence of extramedullary leukemia. Relapse was defined as at least 5% blast cells in the bone marrow or extramedullary leukemia. Leukemia-free survival (LFS) was defined as the time from random assignment to the date of relapse or death from any cause, whichever occurred first. Overall survival was measured from the date of random assignment to death from any cause.

For the present analysis, the trial results were further evaluated as per presence of karyotypic aberrations in leukemic cells (aberrant karyotype) or of absence of karyotypic aberrations (normal karyotype) as defined at diagnosis. For these and any following analysis, only patients in first complete remission (CR1) were considered (n=261). The karyotypic features of leukemic cells were unknown in 14% of patients, and results obtained in 225 patients in CR1 were thus available for analysis of outcome in terms of leukemia-free survival (LFS; defined as the time from random assignment to HDC/IL-2 or control arm to relapse of leukemia or death from any cause) and overall survival (OS; defined as time from random assignment to death from any cause).

Methods to obtain a karyotype of leukemic cells are well known and can include Giemsa-banding which generates a visible karyotype by the staining of condensed chromosomes. See e.g., Speicher and Carter. Nature Reviews Genetics 6:782-792.

### Results

### Subjects Having Non-Aberrant Karyotype

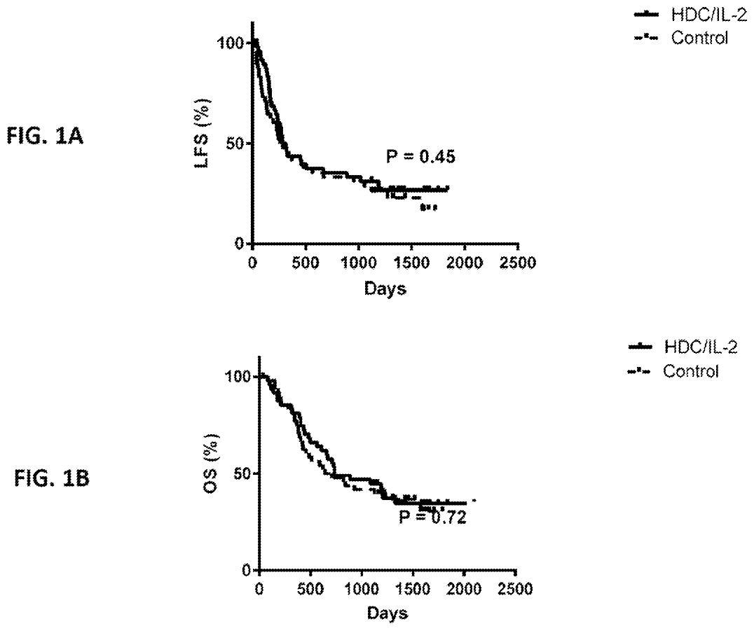

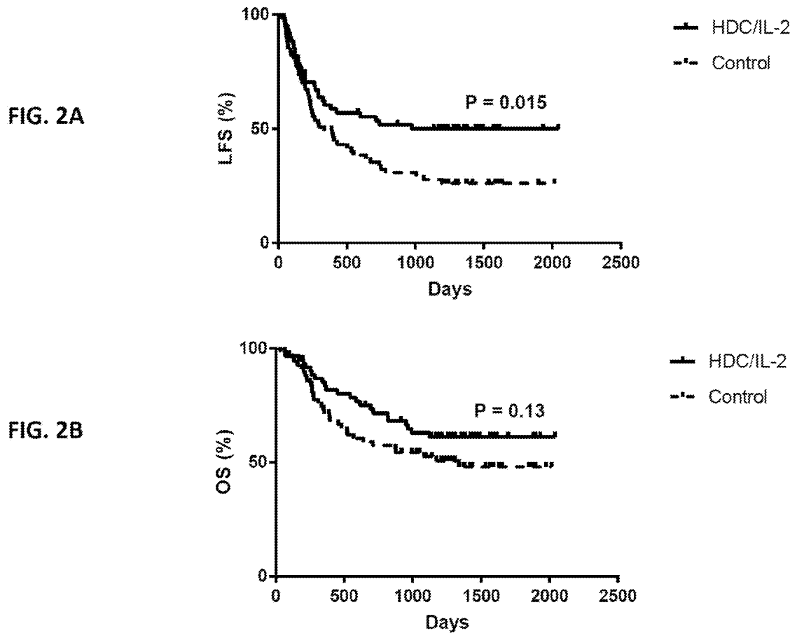

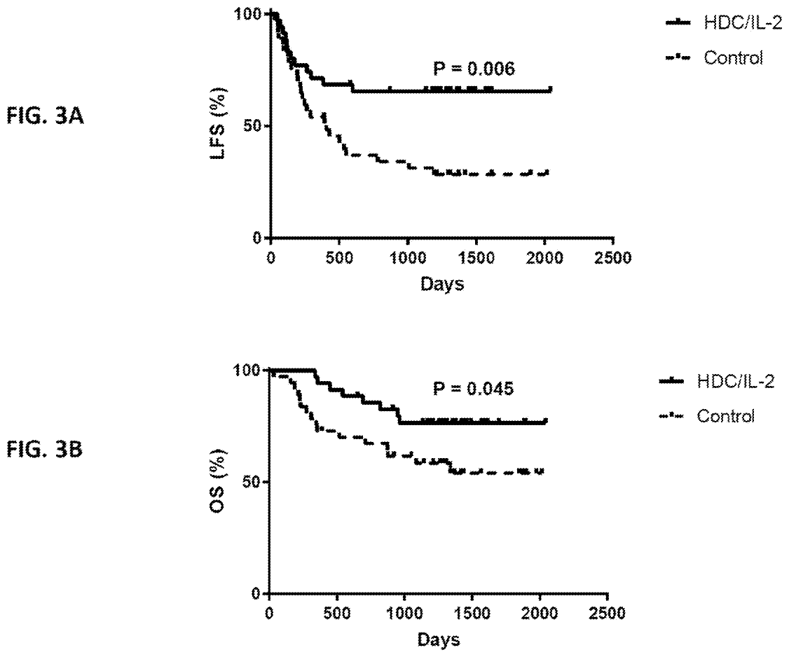

FIG. 1A (LFS) and FIG. 1B (OS) show the outcome of patients diagnosed with AML in their first complete remission (CR1) with structural chromosomal changes in leukemic cells (aberrant karyotype). FIG. 2A and FIG. 2B show corresponding analyses in patients with normal karyotype AML. FIG. 3A and FIG. 3B show the results obtained in the group of younger patients in CR1 with normal karyotype AML. The clinical outcome was compared using the log rank test. The data in FIG. 1-3 demonstrated that the structure of chromosomes in cancer cells from patients with acute myeloid leukemia (AML) determined the clinical efficacy of immunotherapy with histamine dihydrochloride and low-dose interleukin-2 (HDC/IL-2). Therefore, the HDC/IL-2 treatment had unexpectedly high efficacy in normal karyotype AML patients.

Thus, there was an unexpected observation that the structure of chromosomes in cancer cells from patients with acute myeloid leukemia (AML) determines the clinical efficacy of immunotherapy with histamine dihydrochloride and low-dose interleukin-2 (HDC/IL-2). Specifically, it was shown that the leukemia-free survival and overall survival of adult AML patients was favorably impacted by treatment with HDC/IL-2 in patients in whom cancer cells were devoid of structural changes in chromosomes (‘normal karyotype AML’) but clinical efficacy of HDC/IL-2 was absent in cases of AML where any chromosomal alteration was observed (aberrant karyotype).

### Subjects Having a Single Course of Induction Therapy

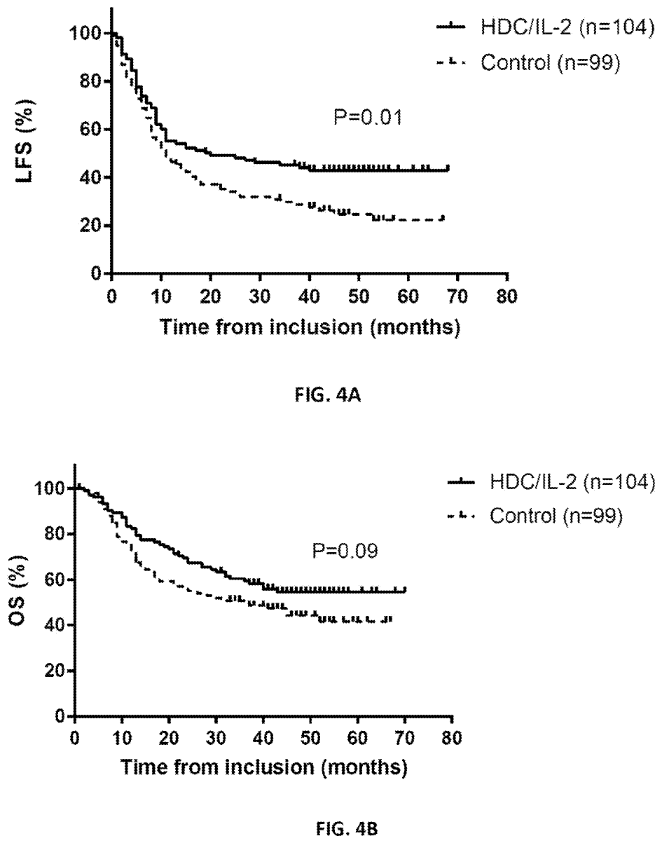

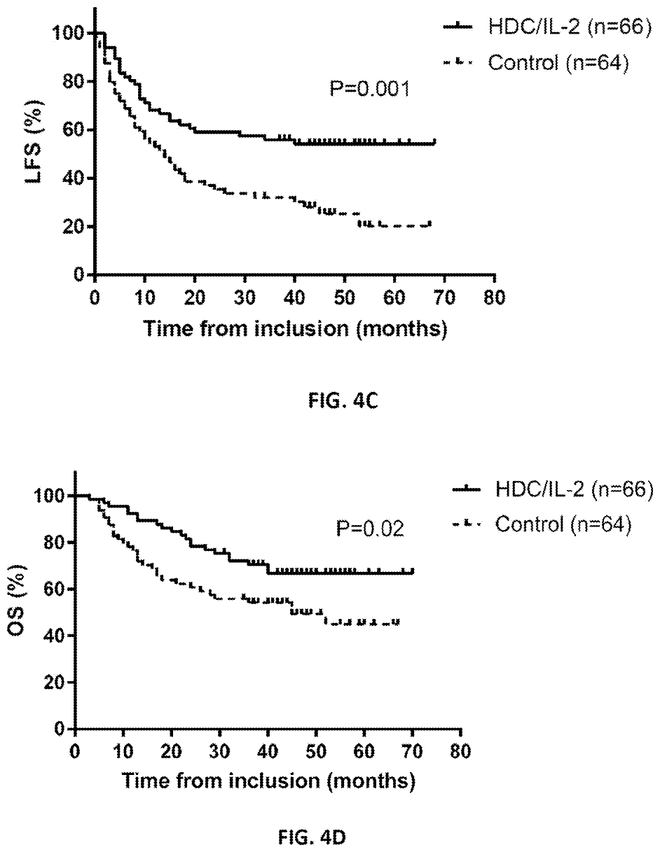

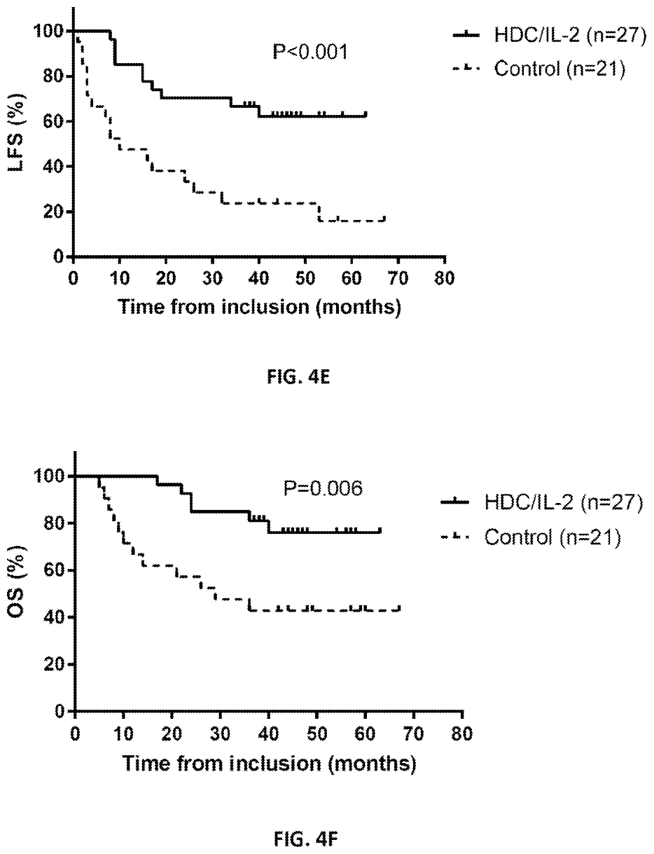

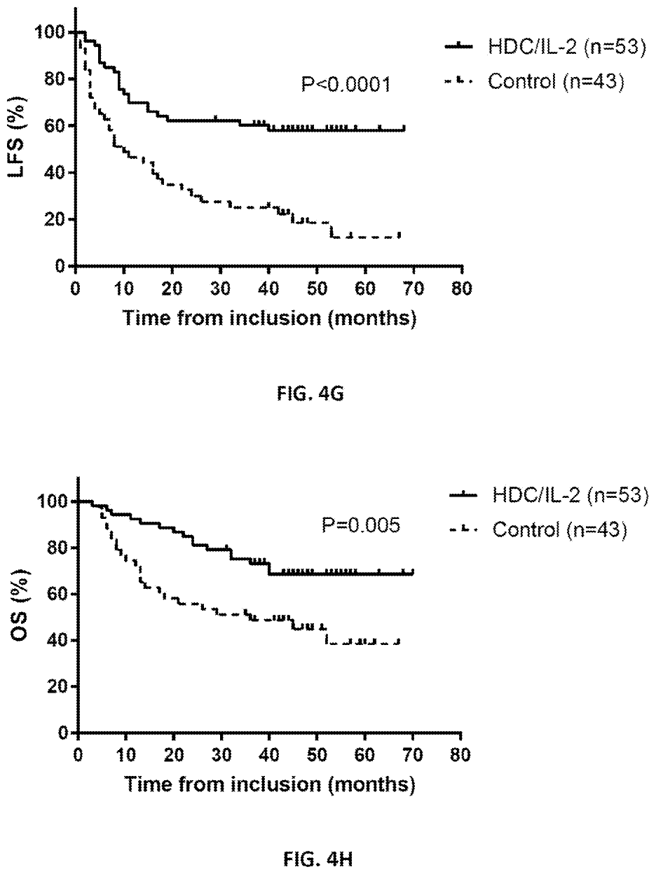

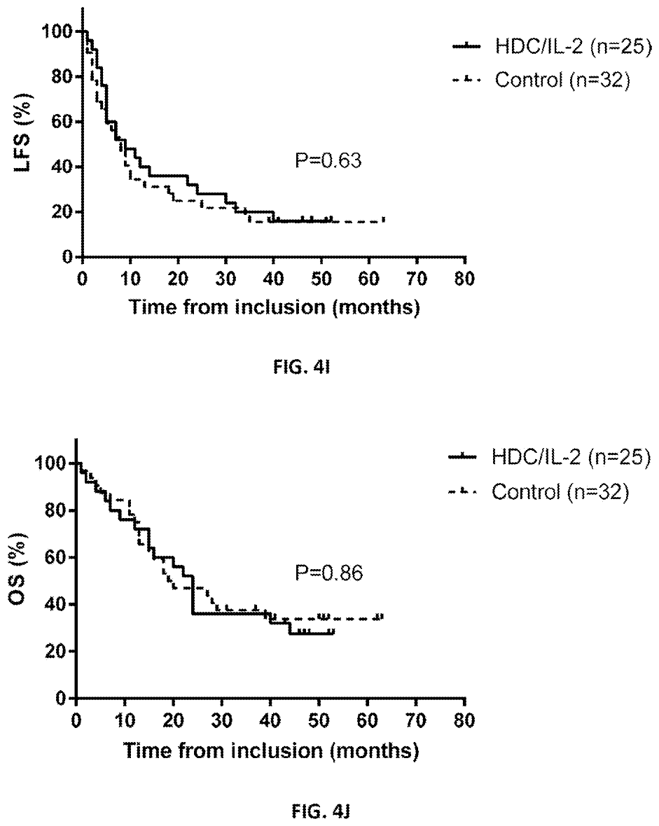

Out of the 261 patients in first complete remission (CR1), 203 patients attained CR1 after a single course of induction chemotherapy, while 57 patients attained CR1 after two or more courses of induction. For one patient in CR1 the number of inductions courses was unknown. The outcome of patients attaining CR1 after a single course of induction is shown by use of Kaplan-Meir statistics in FIG. 4A (leukemia-free survival, LFS) and FIG. 4B (overall survival, OS). In these patients (regardless of age), there was a significant benefit of HDC/IL-2 vs. control treatment for LFS and a non-significant trend towards improved OS.

Corresponding results are shown in the group of patients attaining CR1 after a single course of induction who were <60 years old at trial inclusion in FIG. 4C (LFS) and FIG. 4D (OS). The results thus demonstrated significant clinical benefit of HDC/IL-2 vs. controls for LFS and OS in younger AML patients attaining CR1 after a single course of induction.

FIG. 4E (LFS) and FIG. 4F (OS) show comparisons of outcome in younger (<60 years old) patients with AML of the M4 and M5 French-American-British classification attaining CR1 after a single course of induction. FIG. 4G (LFS) and FIG. 4H (OS) show comparisons of outcome in younger patients with non M2-AML attaining CR1 after a single course of induction, referring to AML of any FAB classification except M2. Thus, the results shown in FIG. 4E-4H show that treatment with HDC/IL-2 was significantly beneficial vs. control patients in terms of LFS and OS in patients with AML of FAB-class M4/M5 and FAB-class non-M2.

Fifty-seven patients (57/260, 22%) needed more than one course of induction chemotherapy to attain CR1 (n=32 in the control group of patients and n=25 in the HDC/IL-2 group). The outcome of these patients is shown by use of Kaplan-Meir statistics in FIG. 4I (leukemia-free survival, LFS) and FIG. 4J (overall survival, OS). As shown, there was no discernable benefit of treatment with HDC/IL-2 vs. controls in patients who needed >1 course of induction chemotherapy to attain CR1. Similarly, in patients below the age of 60 who needed >1 course of induction chemotherapy to attain CR1, there was no significant benefit of HDC/IL-2 compared with control treatment (the log rank p-value was 0.48, for a total of 30 patients).

Thus, there was an unexpected observation that the number of courses of chemotherapy required to achieve first complete remission (CR1) (induction chemotherapy) determined the clinical efficacy of immunotherapy with histamine dihydrochloride and low-dose interleukin 2 (HDC/IL-2). Specifically, it was shown that the leukemia-free survival and overall survival of adult AML patients was favorably impacted by treatment with HDC/IL-2 in patients who attained CR1 after a single course of induction chemotherapy.

The term “comprising” as used herein is synonymous with “including,” “containing,” or “characterized by,” and is inclusive or open-ended and does not exclude additional, unrecited elements or method steps.

The above description discloses several methods and materials. The inventive aspects and embodiments are susceptible to modifications in the methods and materials, as well as alterations in the fabrication methods and equipment. Such modifications will become apparent to those skilled in the art from a consideration of this disclosure or practice of the embodiments disclosed herein. Consequently, it is not intended that this disclosure be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A method of reducing the risk of relapse for a hyperproliferative disorder in a subject, wherein a hyperproliferative cell of the subject comprises a normal karyotype, the method comprising:
administering to the subject an effective amount of a nicotinamide adenine dinucleotide phosphate oxidase 2 (NOX2) inhibitor in combination with an effective amount of a cytokine,
wherein the risk of relapse in the subject is reduced compared to an untreated subject not administered the NOX2 inhibitor in combination with the cytokine.

2. A method of increasing a survival rate of a subject having a hyperproliferative disorder, wherein a hyperproliferative cell of the subject comprises a normal karyotype, the method comprising:
administering to the subject an effective amount of a nicotinamide adenine dinucleotide phosphate oxidase 2 (NOX2) inhibitor in combination with an effective amount of a cytokine,
wherein the survival rate of the subject is increased compared to an untreated subject not administered the NOX2 inhibitor in combination with the cytokine.

3. A method of reducing the risk of relapse for a hyperproliferative disorder in a subject, wherein the subject has been administered no more than one induction chemotherapy course to treat the hyperproliferative disorder, the method comprising:
administering to the subject an effective amount of a nicotinamide adenine dinucleotide phosphate oxidase 2 (NOX2) inhibitor in combination with an effective amount of a cytokine,
wherein the risk of relapse in the subject is reduced compared to an untreated subject not administered the NOX2 inhibitor in combination with the cytokine.

4. A method of increasing a survival rate of a subject having a hyperproliferative disorder, wherein the subject has been administered no more than one induction chemotherapy course to treat the hyperproliferative disorder, the method comprising:
administering to the subject an effective amount of a nicotinamide adenine dinucleotide phosphate oxidase 2 (NOX2) inhibitor in combination with an effective amount of a cytokine,
wherein the survival rate of the subject is increased compared to an untreated subject not administered the NOX2 inhibitor in combination with the cytokine.

5. The method of any one of claims 1-4, wherein the subject is in complete remission (CR) from the hyperproliferative disorder.

6. The method of claim 5, wherein the CR comprises less than 5% blast cells in normocellular bone marrow and an absence of extramedullary leukemia.

7. The method of any one of claims 1-6, wherein the NOX2 inhibitor is selected from the group consisting of histamine, a histamine salt, histamine dihydrochloride (HDC), histamine diphosphate, a histamine structural analog having H2-receptor activities, an endogenous histamine releasing preparation, a non-histamine derivative H2-receptor agonist, GSK2795039, apocynin, diphenylene iodonium, GKT136901, GKT137831, ML171, VAS2870, VAS3947, celastrol, ebselen, perhexiline, grindelic acid, NOX2ds-tat, NOXA1ds, fulvene-5, ACD084, NSC23766, CAS 1177865-17-6, CAS 1090893-12-1, and shionogi.

8. The method of any one of claims 1-7, wherein the NOX2 comprises histamine dihydrochloride (HDC).

9. The method of claim 8, wherein the HDC is administered at a dosage of about 0.5 mg twice a day.

10. The method of any one of claims 1-9, wherein the cytokine is selected from the group consisting of interleukin-2 (IL-2), modified variants of IL-2, interleukin-15 (IL-15), interleukin-18 (IL-18), interferon-alpha, interferon-beta, and interferon-gamma.

11. The method of claim 10, wherein the cytokine is IL-2.

12. The method of claim 11, wherein the IL-2 is administered in an amount of about 5,000 U/kg/day to about 300,000 U/kg/day.

13. The method of claim 12, wherein the IL-2 is administered at a dosage of 16,400 U/kg twice a day.

14. The method of any one of claim 1-13, wherein the NOX2 inhibitor and the cytokine are administered sequentially.

15. The method of claim 14, wherein the NOX2 inhibitor is administered prior to the cytokine.

16. The method of claim 14, wherein the NOX2 inhibitor is administered after the cytokine.

17. The method of any one of claim 1-13, wherein the NOX2 inhibitor and the cytokine are administered concurrently.

18. The method of any one of claim 1-17, wherein the administration comprises a first cycle comprising daily administration of the NOX2 inhibitor in combination of the cytokine for 3 weeks, and no administration of the NOX2 inhibitor in combination of the cytokine for the following 3 weeks.

19. The method of claim 18, wherein the first cycle is repeated for three cycles.

20. The method of claim 18 or 19, further comprising a second cycle comprising daily administration of the NOX2 inhibitor in combination of the cytokine for 3 weeks, and no administration of the NOX2 inhibitor in combination of the cytokine for the following 6 weeks

21. The method of claim 20, wherein the second cycle is repeated from six cycles.

22. The method of any one of claims 1-21, wherein the hyperproliferative disorder is a leukemia.

23. The method of any one of claims 1-22, wherein the hyperproliferative disorder is selected from the group consisting of chronic myeloid leukemia, acute myeloid leukemia (AML), acute lymphocytic leukemia, T-cell acute lymphoblastic leukemia (T-ALL), and chronic lymphocytic leukemia.

24. The method of any one of claims 1-23, wherein the hyperproliferative disorder is acute myeloid leukemia (AML).

25. The method of any one of claims 1-24, wherein a hyperproliferative cell of the subject comprises a normal karyotype.

26. The method of claim 25, wherein the normal karyotype lacks at least a chromosomal duplication, a chromosomal deletion, a chromosomal insertion, a chromosomal inversion, or a chromosomal translocation.

27. The method of claim 25 or 26, wherein the normal karyotype lacks at least one chromosomal aberration selected from a chromosome 5 deletion, a chromosome 7 deletion, a chromosome 8 duplication, a chromosome 21 duplication, a chromosome 22 duplication, a del(5q) deletion, a del(7q) deletion, a del(9q) deletion, a t(8;21) translocation, a t(9;11) translocation, a t(15;17) translocation, and an inv(16) inversion.

28. The method of any one of claims 1-27, further comprising identifying a normal karyotype in the hyperproliferative cell of the subject.

29. The method of claim 28, wherein the identification comprises obtaining a chromosomal spread of the hyperproliferative cell of the subject.

30. The method of claim 28 or 29, wherein the identification comprises performing fluorescent in situ hybridization (FISH) on the hyperproliferative cell of the subject.

31. The method of any one of claims 1-30, wherein administration of the NOX2 inhibitor in combination of the cytokine increases the survival rate of the subject by at least 10% compared to a survival rate for the untreated subject.

32. The method of any one of claims 1-31, wherein administration of the NOX2 inhibitor in combination of the cytokine increases the survival rate of the subject by at least 30% compared to a survival rate for the untreated subject.

33. The method of any one of claims 1-32, wherein administration of the NOX2 inhibitor in combination of the cytokine increases the survival rate of the subject by at least 50% compared to a survival rate for the untreated subject.

34. The method of any one of claims 1-33, wherein administration of the NOX2 inhibitor in combination of the cytokine increases the survival rate of the subject compared to a survival rate for the untreated subject, wherein the survival rate is a leukemia-free survival rate.

35. The method of any one of claims 1-34, wherein administration of the NOX2 inhibitor in combination of the cytokine increases the survival rate of the subject compared to a survival rate for the untreated subject, wherein the survival rate is an overall survival rate.

36. The method of any one of claims 1-35, wherein administration of the NOX2 inhibitor in combination of the cytokine delays relapse of the hyperproliferative disorder in the subject by at least 3 months compared to a delay in relapse for the untreated subject.

37. The method of any one of claims 1-36, wherein administration of the NOX2 inhibitor in combination of the cytokine delays relapse of the hyperproliferative disorder in the subject by at least 6 months compared to a delay in relapse for the untreated subject.

38. The method of any one of claims 1-37, wherein administration of the NOX2 inhibitor in combination of the cytokine delays relapse of the hyperproliferative disorder in the subject by at least 12 months compared to a delay in relapse for the untreated subject.

39. The method of any one of claims 1-38, wherein administration of the NOX2 inhibitor in combination of the cytokine reduces the risk of relapse of the hyperproliferative disorder for the subject by at least 10% compared to a risk of relapse for the untreated subject.

40. The method of any one of claims 1-39, wherein administration of the NOX2 inhibitor in combination of the cytokine reduces the risk of relapse of the hyperproliferative disorder for the subject by at least 30% compared to a risk of relapse for the untreated subject.

41. The method of any one of claims 1-40, wherein administration of the NOX2 inhibitor in combination of the cytokine reduces the risk of relapse of the hyperproliferative disorder for the subject by at least 50% compared to a risk of relapse for the untreated subject.

42. The method of any one of claims 1-41, wherein a relapse of the hyperproliferative disorder comprises at least 5% blast cells in the bone marrow.

43. The method of any one of claims *-*, wherein a relapse of the hyperproliferative disorder comprises extramedullary leukemia.

44. The method of any one of claims 1-43, wherein the subject has been administered no more than one induction chemotherapy course to treat the hyperproliferative disorder.

45. The method of any one of claims 1-44, wherein the subject has not been administered a consolidation therapy.

46. The method of any one of claims 1-45, wherein the subject is less than 60 years old.

47. The method of any one of claims 1-46, wherein the subject is mammalian.

48. The method of any one of claims 1-47, wherein the subject is human.

49. A kit for reducing the risk of relapse for a hyperproliferative disorder in a subject or for increasing a survival rate of the subject, comprising:
a nicotinamide adenine dinucleotide phosphate oxidase 2 (NOX2) inhibitor;
a cytokine; and
a probe to identify a normal karyotype in a hyperproliferative cell.

50. The kit of claim 49, wherein the NOX2 inhibitor is selected from the group consisting of histamine, a histamine salt, histamine dihydrochloride (HDC), histamine diphosphate, a histamine structural analog having H2-receptor activities, an endogenous histamine releasing preparation, a non-histamine derivative H2-receptor agonist, GSK2795039, apocynin, diphenylene iodonium, GKT136901, GKT137831, ML171, VAS2870, VAS3947, celastrol, ebselen, perhexiline, grindelic acid, NOX2ds-tat, NOXA1ds, fulvene-5, ACD084, NSC23766, CAS 1177865-17-6, CAS 1090893-12-1, and shionogi;
wherein the cytokine is selected from the group consisting of interleukin-2 (IL-2), interleukin-15 (IL-15), interleukin-18 (IL-18), interferon-alpha, interferon-beta, and interferon-gamma; and
wherein the probe to identify a normal karyotype in a hyperproliferative cell is selected from a chromosome-specific probe, or a chromosome stain selected from giemsa, mepacrine, DAPI and Hoescht 33258.
